(19) United States
(12) Patent Application Publication
(10) Pub. No.: US 2024/0285331 A1
(43) Pub. Date: Aug. 29, 2024

(54) SYSTEMS AND METHODS FOR POWER CONTROL FOR A THERAPEUTIC TOOL

(71) Applicant: Maquet Cardiovascular LLC, Wayne, NJ (US)

(72) Inventors: Juan Isaias Perez, San Jose, CA (US); Ashik A. Mohan, Alamo, CA (US); Samuel R. Phillips, San Mateo, CA (US); Marc S. Kreidler, Sunnyvale, CA (US); John Tosto, Ramsey, NJ (US); Maw-Lin Kuo, Fremont, CA (US); Tachi Callas, Castro Valley, CA (US)

(73) Assignee: Maquet Cardiovascular LLC, Wayne, NJ (US)

(21) Appl. No.: 18/585,958

(22) Filed: Feb. 23, 2024

Related U.S. Application Data
(60) Provisional application No. 63/486,870, filed on Feb. 24, 2023.

(51) Int. Cl. A61B 18/10; A61B 17/00; A61B 18/00; A61B 18/08; H05K 7/14

(52) U.S. Cl. CPC A61B 18/10; A61B 18/085; H05K 7/1427; A61B 2017/00969; A61B 2018/00428; A61B 2018/00595; A61B 2018/00601; A61B 2018/00619; A61B 2018/00702

(57) ABSTRACT

Provided is an endoscopic vessel harvesting system comprising a direct current (DC) power control system connected to a therapeutic tool comprising a heating element. The DC power control system may include an input connection to receive power from a power supply. A first power control circuit may supply constant output power during a first time interval to heat the heating element to a target temperature. A second power control circuit may supply pulsed output power during a second time interval to maintain a temperature of the heating element within a target temperature range. An output connection may receive the output power and may supply controlled power to the heating element by supplying the constant output power during the first time interval, the pulsed output power during the second time interval, followed by a third time interval during which no power is supplied. Methods and systems are also disclosed.

### CROSS-REFERENCE TO RELATED APPLICATION

This application claims priority to U.S. Provisional Patent Application No. 63/486,870, filed Feb. 24, 2023, the disclosure of which is hereby incorporated by reference in its entirety.

### BACKGROUND

### 1. Technical Field

This disclosed subject matter relates generally to systems and methods for power control and, in some non-limiting embodiments or aspects, to systems and methods for power control for a therapeutic tool.

### 2. Technical Considerations

Certain therapeutic tools, such as ablation devices, cutting devices, cauterizing devices, and/or endoscopic vessel harvesting (EVH) devices, use heating elements, for example, to assist in cutting tissue, to cauterize tissue, and/or to ablate tissue. When power is provided to such a heating element, the temperature of the heating element rises. If the temperature is properly controlled, such heating elements may be advantageous for certain medical procedures, such as cutting, ablating, cauterizing, and/or vessel harvesting.

However, if the temperature of such heating elements becomes excessively high, such excessive temperatures may collaterally damage adjacent tissues and/or nearby tissues and/or may damage the therapeutic tool and/or components thereof. For example, if the temperature is too high, the heat may unnecessarily damage tissue surrounding the target tissue at which the procedure is directed, whether the unnecessarily damaged tissue is contiguous with the target tissue or located near to the target tissue (but not necessarily contiguous with it). Excessive heat may damage certain components, such as semiconductor components, of the therapeutic tool and/or may cause melting and/or burning of certain materials of the therapeutic tool. Furthermore, if the temperature is insufficiently low, the heating element may be ineffective and/or inefficient when performing the intended function thereof, such as assisting in or directly cutting, ablating, and/or cauterizing tissue.

Certain therapeutic tools may include safeguards, such as a fuse or a polyfuse, to prevent overheating. For example, a polyfuse may be intended to turn the power off at a certain level of power delivery. However, the power limit may not be constant. For example, the power limit may fluctuate based on environmental factors, may be unreliable, and/or may have an undesirably wide range. The polyfuse may trip (turn off power) after an unreliable amount of time, for example, too short of an amount of time (e.g., 2 seconds) to perform a procedure or too long of an amount of time (e.g., 18 seconds) to prevent overheating. Consequently, the heating element of the therapeutic tool may still become too hot, thereby damaging and/or breaking the device and/or damaging collateral tissue. Also, the amount of time for the polyfuse to reset may not be predictable and/or reliable, and, therefore, the polyfuse may require more time to reset than desired before allowing additional and/or subsequent procedures to be performed.

Certain users may attempt to manually turn off power to the therapeutic tool using a switch or toggle, for example, which may be on the handle of the therapeutic tool. However, the temperature at which power is turned off and/or the speed of the user reacting to overheating may not be constant and/or reliable. Consequently, the user may still inadvertently allow overheating and/or under-heating to occur. Additionally, it may be uncomfortable and/or distracting to the user to be toggling a switch manually rather than focusing on the procedure performed.

### SUMMARY

Accordingly, it is an object of the presently disclosed subject matter to provide systems and methods for power control for a therapeutic tool that overcome some or all of the deficiencies identified above.

According to non-limiting embodiments or aspects, provided is an endoscopic vessel harvesting system. An endoscopic vessel harvesting system may include a direct current power control system connected to provide controlled power to a therapeutic tool, which may include a heating element. The direct current power control system may include an input connection, which may be configured to receive power from a power supply. The direct current power control system may include a first power control circuit connected to the input connection. The first power control circuit may be configured to supply constant output power to the heating element during a first time interval to heat the heating element to a target temperature. The direct current power control system may include a second power control circuit connected to the input connection. The second power control circuit may be configured to supply pulsed output power to the heating element during a second time interval following the first time interval to maintain a temperature of the heating element within a target temperature range. The direct current power control system may include an output connection connected to the first power control circuit and the second power control circuit. The output connection may be configured to receive the constant output power from the first power control circuit and the pulsed output power from the second power control circuit to supply controlled power to the heating element of the therapeutic tool. Supplying of controlled power to the heating element may include, in sequence, supplying the constant output power during the first time interval and then the pulsed output power during the second time interval to the heating element followed by a third time interval during which no power is supplied to the heating element to allow heat to dissipate from the heating element.

In some non-limiting embodiments or aspects, the first power control circuit may include a first one-shot pulse generator circuit.

In some non-limiting embodiments or aspects, the first time interval may include a time interval greater than or equal to 2 seconds and less than or equal to 10 seconds so that the heating element heats to the target temperature effective for performing an endoscopic vessel harvesting procedure.

In some non-limiting embodiments or aspects, the target temperature effective for performing the endoscopic vessel harvesting procedure may include a first temperature sufficient to at least one of cut, cauterize, or weld target tissue.

In some non-limiting embodiments or aspects, the second power control circuit may include a second one-shot pulse generator circuit and an oscillator circuit. An output of the second one-shot pulse generator circuit may be connected to an input of the oscillator circuit.

In some non-limiting embodiments or aspects, the second time interval may include a time interval greater than or equal to 5 seconds and less than or equal to 20 seconds.

In some non-limiting embodiments or aspects, the second time interval may be selected based on an average time to at least one of cut, cauterize, or weld target tissue.

In some non-limiting embodiments or aspects, the pulsed output power may have a frequency of greater than or equal to 2.5 Hz and less than or equal to 12 Hz.

In some non-limiting embodiments or aspects, the frequency may be selected to maintain the temperature of the heating element at the target temperature range effective for performing an endoscopic vessel harvesting procedure.

In some non-limiting embodiments or aspects, the system further may include a circuit board, which may include the first power control circuit and the second power control circuit. In some non-limiting embodiments or aspects, the system further may include a housing containing the circuit board and the output connection. In some non-limiting embodiments or aspects, the system further may include a cable extending from the housing, the cable having a proximal end connected to the input connection and a distal end connected to the circuit board.

In some non-limiting embodiments or aspects, the system further may include a first logic gate connected to the first power control circuit and the second power control circuit. The first logic gate may be configured to output only one of the constant output power from the first power control circuit or the pulsed output power from the second power control circuit at a time. In some non-limiting embodiments or aspects, the system further may include a second logic gate connected to the first logic gate and a switch. The second logic gate may be configured to output the constant output power or the pulsed output power from the first logic gate only after the switch is closed.

According to non-limiting embodiments or aspects, provided is a method for providing controlled power via a direct current power control system of an endoscopic vessel harvesting device so as to provide controlled power to a heating element of a therapeutic tool of the endoscopic vessel harvesting device. The method may include receiving, at an input connection of the direct current power control system, power from a power supply. The method may include supplying power from the input connection to a first power control circuit and a second power control circuit of the direct current power control system. The method may include supplying constant output power from the first power control circuit during a first time interval to heat the heating element to a target temperature. The method may include supplying pulsed output power from the second power control circuit during a second time interval following the first time interval to maintain a temperature of the heating element within a target temperature range. The method may include receiving, at an output connection of the direct current power control system, the constant output power from the first power control circuit or the pulsed output power from the second power control circuit. The method may include supplying the constant output power or the pulsed output power from the output connection to the therapeutic tool. The method may include supplying no power to the heating element during a third time interval following the second time interval to allow heat to dissipate from the heating element.

In some non-limiting embodiments or aspects, the first time interval may include a time interval greater than or equal to 2 seconds and less than or equal to 10 seconds so that the heating element heats to the target temperature effective for performing an endoscopic vessel harvesting procedure.

In some non-limiting embodiments or aspects, the target temperature effective for performing the endoscopic vessel harvesting procedure may include a first temperature sufficient to at least one of cut, cauterize, or weld target tissue.

In some non-limiting embodiments or aspects, the second time interval may include a time interval greater than or equal to 5 seconds and less than or equal to 20 seconds.

In some non-limiting embodiments or aspects, the second time interval may be selected based on an average time to at least one of cut, cauterize, or weld target tissue.

In some non-limiting embodiments or aspects, the pulsed output power has a frequency of greater than or equal to 2.5 Hz and less than or equal to 12 Hz.

In some non-limiting embodiments or aspects, the frequency may be selected to maintain the temperature of the heating element at the target temperature range effective for performing an endoscopic vessel harvesting procedure.

According to non-limiting embodiments or aspects, provided is a method for making a direct current power control system for an endoscopic vessel harvesting system to provide controlled power to a therapeutic tool may include a heating element. The method may include connecting an input connection to a circuit board, which may include a first power control circuit and a second power control circuit. The input connection may be configured to receive power from a power supply. The first power control circuit may be configured to supply constant output power to the heating element during a first time interval to heat the heating element to a target temperature. The second power control circuit may be configured to supply pulsed output power to the heating element during a second time interval following the first time interval to maintain a temperature of the heating element within a target temperature range. The method may include connecting the circuit board to an output connection. The output connection may be configured to receive the constant output power and the pulsed output power to supply controlled power to the heating element of the therapeutic tool. Supplying of controlled power to the heating element may include, in sequence, supplying the constant output power during the first time interval and then the pulsed output power during the second time interval to the heating element followed by a third time interval during which no power is supplied to the heating element to allow heat to dissipate from the heating element.

In some non-limiting embodiments or aspects, the first power control circuit may include a first one-shot pulse generator circuit.

In some non-limiting embodiments or aspects, the second power control circuit may include a second one-shot pulse generator circuit and an oscillator circuit. An output of the second one-shot pulse generator circuit may be connected to an input of the oscillator circuit.

In some non-limiting embodiments or aspects, the method further may include connecting the first power control circuit and the second power control circuit to the circuit board before connecting the input connection to the circuit board.

In some non-limiting embodiments or aspects, the method further may include encasing the circuit board and the output connection in a housing. Connecting the input connection to the circuit board may include connecting the circuit board to a distal end of a cable extending from the housing and connecting the input connection to a proximal end of the cable.

In some non-limiting embodiments or aspects, the method further may include connecting a first logic gate to the circuit board so that the first logic gate is connected to the first power control circuit and the second power control circuit. The first logic gate may be configured to output only one of the constant output power from the first power control circuit or the pulsed output power from the second power control circuit at a time. In some non-limiting embodiments or aspects, the method further may include connecting a second logic gate to the circuit board so that the second logic gate is connected to the first logic gate and a switch. The second logic gate may be configured to output the constant output power or the pulsed output power from the first logic gate only after the switch is closed.

According to non-limiting embodiments or aspects, provided is an endoscopic vessel harvesting system. The endoscopic vessel harvesting system may include a power supply, a therapeutic tool, and a direct current power control system. The therapeutic tool may include a heating element connected to a cutting element. The direct current power control system may include an input connection, which may be configured to receive power from the power supply. The direct current power control system may include a circuit board connected to the input connection. The circuit board may be configured to supply constant output power to the heating element during a first time interval to heat the heating element to a target temperature and to supply pulsed output power to the heating element during a second time interval following the first time interval to maintain a temperature of the heating element within a target temperature range. The direct current power control system may include an output connection connected to the circuit board. The output connection may be configured to receive one of the constant output power or the pulsed output power at a time and/or to supply controlled power to the heating element. Supplying of controlled power to the heating element may include, in sequence, supplying the constant output power during the first time interval and then the pulsed output power during the second time interval to the heating element followed by a third time interval during which no power is supplied to the heating element in order to allow heat to dissipate from the heating element to avoid overheating the heating element.

In some non-limiting embodiments or aspects, the circuit board may include a first power control circuit connected to the input connection. The first power control circuit may be configured to supply the constant output power to the heating element during the first time interval to heat the heating element to the target temperature. In some non-limiting embodiments or aspects, the circuit board may include a second power control circuit connected to the input connection. The second power control circuit may be configured to supply the pulsed output power to the heating element during the second time interval to maintain a temperature of the heating element within a target temperature range.

Other non-limiting embodiments or aspects will be set forth in the following numbered clauses:

Clause 1: An endoscopic vessel harvesting system comprising a direct current power control system connected to provide controlled power to a therapeutic tool comprising a heating element, the direct current power control system comprising: an input connection configured to receive power from a power supply; a first power control circuit connected to the input connection, the first power control circuit configured to supply constant output power to the heating element during a first time interval to heat the heating element to a target temperature; a second power control circuit connected to the input connection, the second power control circuit configured to supply pulsed output power to the heating element during a second time interval following the first time interval to maintain a temperature of the heating element within a target temperature range; and an output connection connected to the first power control circuit and the second power control circuit, the output connection configured to receive the constant output power from the first power control circuit and the pulsed output power from the second power control circuit to supply controlled power to the heating element of the therapeutic tool, wherein supplying of controlled power to the heating element comprises, in sequence, supplying the constant output power during the first time interval and then the pulsed output power during the second time interval to the heating element followed by a third time interval during which no power is supplied to the heating element to allow heat to dissipate from the heating element.

Clause 2: The endoscopic vessel harvesting system of clause 1, wherein the first power control circuit comprises a first one-shot pulse generator circuit.

Clause 3: The endoscopic vessel harvesting system of clause 1 or clause 2, wherein the first time interval comprises a time interval greater than or equal to 2 seconds and less than or equal to 10 seconds so that the heating element heats to the target temperature effective for performing an endoscopic vessel harvesting procedure.

Clause 4: The endoscopic vessel harvesting system of any of clauses 1-3, wherein the target temperature effective for performing the endoscopic vessel harvesting procedure comprises a first temperature sufficient to at least one of cut, cauterize, or weld target tissue.

Clause 5: The endoscopic vessel harvesting system of any of clauses 1-4, wherein the second power control circuit comprises a second one-shot pulse generator circuit and an oscillator circuit, wherein an output of the second one-shot pulse generator circuit is connected to an input of the oscillator circuit.

Clause 6: The endoscopic vessel harvesting system of any of clauses 1-5, wherein the second time interval comprises a time interval greater than or equal to 5 seconds and less than or equal to 20 seconds.

Clause 7: The endoscopic vessel harvesting system of any of clauses 1-6, wherein the second time interval is selected based on an average time to at least one of cut, cauterize, or weld target tissue.

Clause 8: The endoscopic vessel harvesting system of any of clauses 1-7, wherein the pulsed output power has a frequency of greater than or equal to 2.5 Hz and less than or equal to 12 Hz.

Clause 9: The endoscopic vessel harvesting system of any of clauses 1-8, wherein the frequency is selected to maintain the temperature of the heating element at the target temperature range effective for performing an endoscopic vessel harvesting procedure.

Clause 10: The endoscopic vessel harvesting system of any of clauses 1-9, further comprising: a circuit board comprising the first power control circuit and the second power control circuit; a housing containing the circuit board and the output connection; and a cable extending from the housing, the cable having a proximal end connected to the input connection and a distal end connected to the circuit board.

Clause 11: The endoscopic vessel harvesting system of any of clauses 1-10, further comprising: a first logic gate connected to the first power control circuit and the second power control circuit, wherein the first logic gate is configured to output only one of the constant output power from the first power control circuit or the pulsed output power from the second power control circuit at a time; and a second logic gate connected to the first logic gate and a switch, wherein the second logic gate is configured to output the constant output power or the pulsed output power from the first logic gate only after the switch is closed.

Clause 12: A method for providing controlled power via a direct current power control system of an endoscopic vessel harvesting device so as to provide controlled power to a heating element of a therapeutic tool of the endoscopic vessel harvesting device, the method comprising: receiving, at an input connection of the direct current power control system, power from a power supply; supplying power from the input connection to a first power control circuit and a second power control circuit of the direct current power control system, supplying constant output power from the first power control circuit during a first time interval to heat the heating element to a target temperature; supplying pulsed output power from the second power control circuit during a second time interval following the first time interval to maintain a temperature of the heating element within a target temperature range; receiving, at an output connection of the direct current power control system, the constant output power from the first power control circuit or the pulsed output power from the second power control circuit; supplying the constant output power or the pulsed output power from the output connection to the therapeutic tool; and supplying no power to the heating element during a third time interval following the second time interval to allow heat to dissipate from the heating element.

Clause 13: The method of clause 12, wherein the first time interval comprises a time interval greater than or equal to 2 seconds and less than or equal to 10 seconds so that the heating element heats to the target temperature effective for performing an endoscopic vessel harvesting procedure.

Clause 14: The method of clause 12 or clause 13, wherein the target temperature effective for performing the endoscopic vessel harvesting procedure comprises a first temperature sufficient to at least one of cut, cauterize, or weld target tissue.

Clause 15: The method of any of clauses 12-14, wherein the second time interval comprises a time interval greater than or equal to 5 seconds and less than or equal to 20 seconds.

Clause 16: The method of any of clauses 12-15, wherein the second time interval is selected based on an average time to at least one of cut, cauterize, or weld target tissue.

Clause 17: The method of any of clauses 12-16, wherein the pulsed output power has a frequency of greater than or equal to 2.5 Hz and less than or equal to 12 Hz.

Clause 18: The method of any of clauses 12-17, wherein the frequency is selected to maintain the temperature of the heating element at the target temperature range effective for performing an endoscopic vessel harvesting procedure.

Clause 19: A method for making a direct current power control system for an endoscopic vessel harvesting system to provide controlled power to a therapeutic tool comprising a heating element, the method comprising: connecting an input connection to a circuit board comprising a first power control circuit and a second power control circuit, the input connection configured to receive power from a power supply, the first power control circuit configured to supply constant output power to the heating element during a first time interval to heat the heating element to a target temperature, and the second power control circuit configured to supply pulsed output power to the heating element during a second time interval following the first time interval to maintain a temperature of the heating element within a target temperature range; and connecting the circuit board to an output connection, the output connection configured to receive the constant output power and the pulsed output power to supply controlled power to the heating element of the therapeutic tool, wherein supplying of controlled power to the heating element comprises, in sequence, supplying the constant output power during the first time interval and then the pulsed output power during the second time interval to the heating element followed by a third time interval during which no power is supplied to the heating element to allow heat to dissipate from the heating element.

Clause 20: The method of clause 19, wherein the first power control circuit comprises a first one-shot pulse generator circuit.

Clause 21: The method of clause 19 or clause 20, wherein the second power control circuit comprises a second one-shot pulse generator circuit and an oscillator circuit, wherein an output of the second one-shot pulse generator circuit is connected to an input of the oscillator circuit.

Clause 22: The method of any of clauses 19-21, further comprising connecting the first power control circuit and the second power control circuit to the circuit board before connecting the input connection to the circuit board.

Clause 23: The method of any of clauses 19-22, further comprising: encasing the circuit board and the output connection in a housing, wherein connecting the input connection to the circuit board comprises connecting the circuit board to a distal end of a cable extending from the housing and connecting the input connection to a proximal end of the cable.

Clause 24: The method of any of clauses 19-23, further comprising: connecting a first logic gate to the circuit board so that the first logic gate is connected to the first power control circuit and the second power control circuit, wherein the first logic gate is configured to output only one of the constant output power from the first power control circuit or the pulsed output power from the second power control circuit at a time; and connecting a second logic gate to the circuit board so that the second logic gate is connected to the first logic gate and a switch, wherein the second logic gate is configured to output the constant output power or the pulsed output power from the first logic gate only after the switch is closed.

Clause 25: An endoscopic vessel harvesting system, comprising: a power supply; a therapeutic tool comprising a heating element connected to a cutting element; and a direct current power control system, comprising: an input connection configured to receive power from the power supply; a circuit board connected to the input connection, the circuit board configured to supply constant output power to the heating element during a first time interval to heat the heating element to a target temperature and to supply pulsed output power to the heating element during a second time interval following the first time interval to maintain a temperature of the heating element within a target temperature range; and an output connection connected to the circuit board, the output connection configured to receive one of the constant output power or the pulsed output power at a time, and to supply controlled power to the heating element, wherein supplying of controlled power to the heating element comprises, in sequence, supplying the constant output power during the first time interval and then the pulsed output power during the second time interval to the heating element followed by a third time interval during which no power is supplied to the heating element in order to allow heat to dissipate from the heating element to avoid overheating the heating element.

Clause 26: The system of clause 25, wherein the circuit board comprises: a first power control circuit connected to the input connection, the first power control circuit configured to supply the constant output power to the heating element during the first time interval to heat the heating element to the target temperature; and a second power control circuit connected to the input connection, the second power control circuit configured to supply the pulsed output power to the heating element during the second time interval to maintain a temperature of the heating element within a target temperature range.

These and other features and characteristics of the presently disclosed subject matter, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter. As used in the specification and the claims, the singular form of “a,” “an,” and “the” include plural referents unless the context clearly dictates otherwise.

### BRIEF DESCRIPTION OF THE DRAWINGS

Additional advantages and details of the disclosed subject matter are explained in greater detail below with reference to the exemplary embodiments or aspects that are illustrated in the accompanying figures, in which:

FIGS. 1A-1F are diagrams of non-limiting embodiments or aspects of an environment in which systems and/or methods, described herein, may be implemented according to the principles of the presently disclosed subject matter;

FIG. 2A is a diagram of an exemplary implementation of non-limiting embodiments or aspects of a power control system for a therapeutic tool according to the principles of the presently disclosed subject matter;

FIGS. 2B and 2C are diagrams of an exemplary implementation of non-limiting embodiments or aspects of a power control system for a therapeutic tool according to the principles of the presently disclosed subject matter;

FIG. 3A is a circuit diagram of an exemplary implementation of non-limiting embodiments or aspects of a variable power modulation profile power control system for a therapeutic tool according to the principles of the presently disclosed subject matter;

FIGS. 3B and 3C are circuit diagrams of an exemplary implementation of non-limiting embodiments or aspects of a fixed power modulation profile power control system for a therapeutic tool according to the principles of the presently disclosed subject matter;

FIG. 4A is a graph of current versus time of an exemplary implementation of non-limiting embodiments or aspects of a system and/or method for power control for a therapeutic tool according to the principles of the presently disclosed subject matter;

FIG. 4B is a graph of temperature versus time of an exemplary implementation of non-limiting embodiments or aspects of a system and/or method for power control for a therapeutic tool according to the principles of the presently disclosed subject matter;

FIG. 4C is a graph of temperature and power density versus time of an exemplary implementation of non-limiting embodiments or aspects of a system and/or method for power control for a therapeutic tool according to the principles of the presently disclosed subject matter;

FIG. 5 is a flowchart of non-limiting embodiments or aspects of a process for using a power control system for a therapeutic tool according to the principles of the presently disclosed subject matter;

FIG. 6 is a flowchart of non-limiting embodiments or aspects of a process for making a power control system for a therapeutic tool according to the principles of the presently disclosed subject matter;

FIGS. 7A and 7B are diagrams of non-limiting embodiments or aspects of an exemplary therapeutic tool according to the principles of the presently disclosed subject matter;

FIGS. 8A-8D are diagrams of non-limiting embodiments or aspects of an exemplary therapeutic tool according to the principles of the presently disclosed subject matter;

FIGS. 9A and 9B are diagrams of an exemplary implementation of non-limiting embodiments or aspects of a power control system for a therapeutic tool according to the principles of the presently disclosed subject matter; and

FIGS. 10A and 10B are diagrams of an exemplary implementation of non-limiting embodiments or aspects of a power control system for a therapeutic tool according to the principles of the presently disclosed subject matter.

### DESCRIPTION

For purposes of the description hereinafter, the terms “end,” “upper,” “lower,” “right,” “left,” “vertical,” “horizontal,” “top,” “bottom,” “lateral,” “longitudinal,” “distal,” “proximal,” and derivatives thereof shall relate to the disclosed subject matter as it is oriented in the drawing figures. However, it is to be understood that the disclosed subject matter may assume various alternative variations and step sequences, except where expressly specified to the contrary. It is also to be understood that the specific devices and processes illustrated in the attached drawings, and described in the following specification, are simply exemplary embodiments or aspects of the disclosed subject matter. Hence, specific dimensions and other physical characteristics related to the embodiments or aspects disclosed herein are not to be considered as limiting unless otherwise specifically indicated.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

Non-limiting embodiments or aspects of the disclosed subject matter are directed to systems and methods for power control, including, but not limited to, power control for a therapeutic tool. For example, non-limiting embodiments or aspects of the disclosed subject matter provide at least one circuit (e.g., first and second power control circuits) configured to supply, to a therapeutic tool, constant output power during a first time interval and to supply pulsed output power during a second time interval following the first time interval. Such embodiments provide techniques and systems that provide power to a heating element of a therapeutic tool predictably and reliably. Additionally or alternatively, such embodiments provide techniques and systems that enable heating of the heating element to a target temperature during the first time interval and/or maintaining the temperature of the heating element within a target range during the second time interval. As such, the temperature of the heating element may be maintained at a safe and/or effective temperature during a medical procedure, and overheating and/or under-heating may be prevented. For example, during the first time interval, the constant power may heat the heating element to an effective temperature, and during the second time interval, the pulsed power may maintain the temperature in an effective range while preventing overheating, thereby preventing damage to the therapeutic tool and/or unnecessary damage to collateral tissue. Additionally or alternatively, such embodiments provide techniques and systems that enable cessation of power after the second time interval such that, during a third time interval following the second time interval, no power may be provided. Consequently, after the second time interval, the heating element may no longer receive power and the temperature thereof may decrease, thus preventing heating of the heating element for too long. For example, after sufficient time to perform a medical procedure, the power is cut off to prevent damage to the device and/or collateral tissue from inadvertently allowing power to be delivered to the heating element for too long and/or after the procedure has been adequately completed. Furthermore, after the aforementioned time intervals are complete, the user may use the power control system and therapeutic tool connected thereto again by simply starting again, such as by toggling a switch, without needing to wait for an unreliable/unpredictable failsafe mechanism, such as a polyfuse, to reset. Additionally or alternatively, such embodiments provide techniques and systems that enable automated control of the temperature of the heating element of the therapeutic tool without requiring the user to manually turn off the power and/or monitor the temperature. As such, at least one of potential human errors, such as inconsistency in the temperature at which power is turned off, inconsistency in the speed of the user reacting to overhearing, discomfort of the user, or distractions to the user may be avoided because a user need not repeatedly manually toggle a power switch during a medical procedure in order to avoid overheating the heating element and/or in order to avoid heating of the heating element beyond what is needed to successfully complete a medical procedure without damaging the therapeutic tool or adjacent tissues or collateral tissues.

For the purpose of illustration, in the following description, while the presently disclosed subject matter is described with respect to systems and methods for power control for a therapeutic tool, one skilled in the art will recognize that the disclosed subject matter is not limited to the illustrative embodiments or aspects.

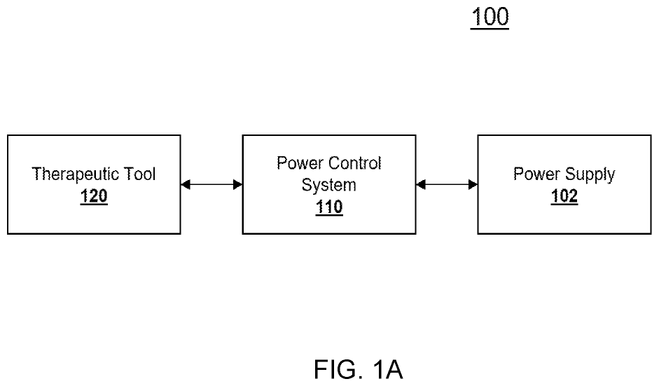

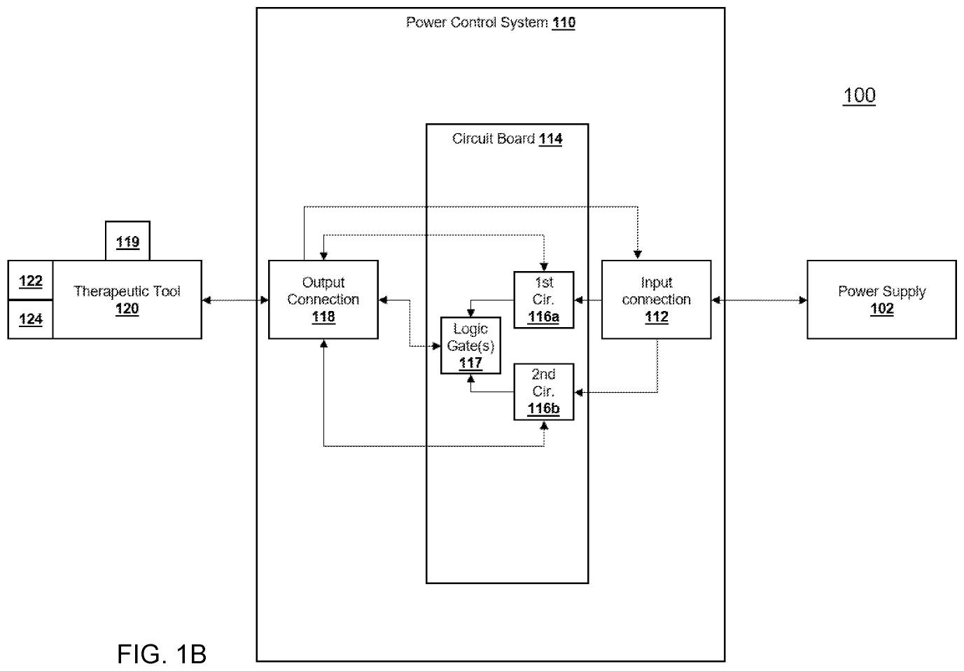

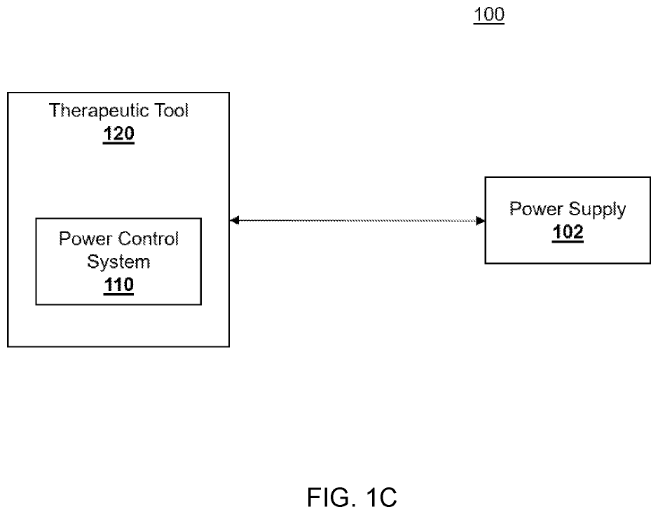

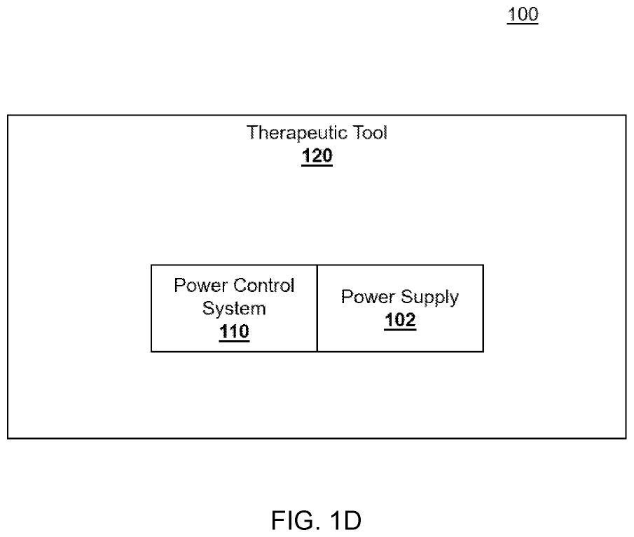

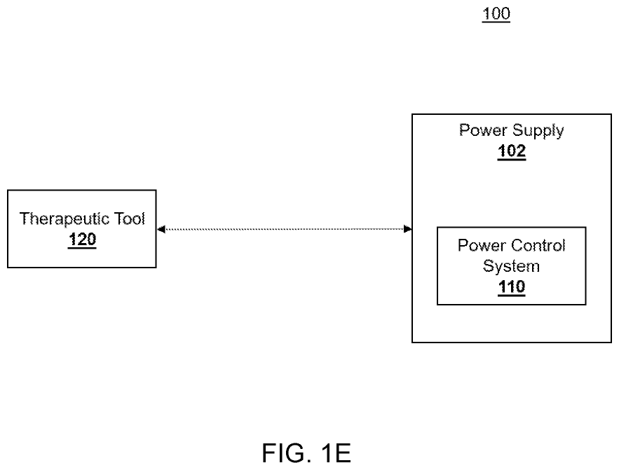

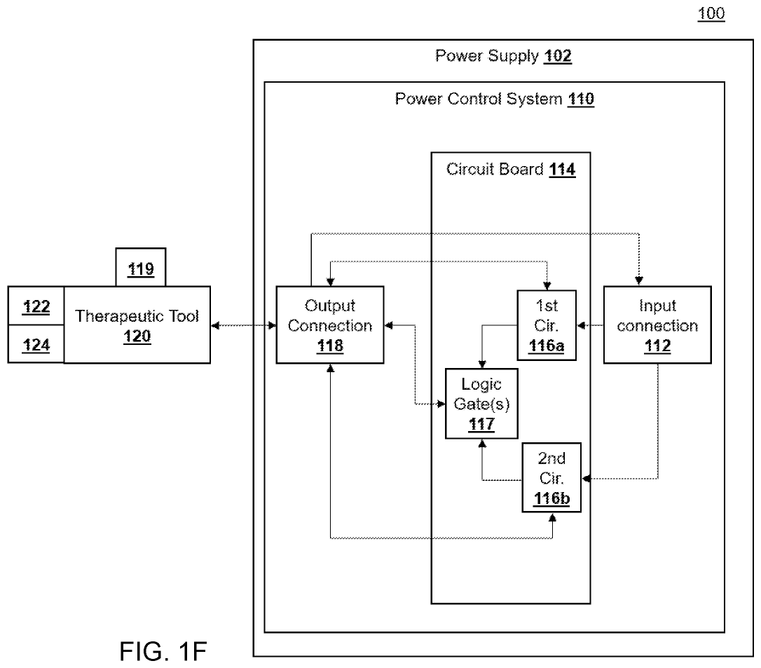

FIGS. 1A-1F are diagrams of non-limiting embodiments or aspects of an environment 100 in which systems and/or methods, as described herein, may be implemented. As shown in FIGS. 1A-1F, environment 100 may include power supply 102, power control system 110 (including input connection 112, circuit board 114, first power control circuit 116a, second power control circuit 116b, logic gate(s) 117, and/or output connection 118), and/or therapeutic tool 120 (including heating element 122, sensor 124, and/or switch 119). Environment 100 may be configured so that power supply 102, power control system 110, and therapeutic tool 120 may be separate components of environment 100 that are operably connected to one another (e.g., as shown in FIG. 1A). In another configuration of environment 100, power control system 110 may be incorporated within therapeutic tool 120, and power supply 102 may be operably connected to provide power as an external power supply, such as an external battery pack or an energized electrical socket, to power control system 110 and therapeutic tool 120 (e.g., as shown in FIG. 1C). In another configuration of environment 100, both power control system 110 and power supply 102 are incorporated within the housing of the therapeutic tool 120 (e.g., as shown in FIG. 1D). In this configuration, power supply 102 may constitute an internal battery or battery pack or other suitable portable power source. In another configuration of environment 100, power control system 110 may be incorporated within power supply 102, and power supply 102 (and/or power control system 110) may be operably connected to provide power as an external power supply to therapeutic tool 120 (e.g., as shown in FIGS. 1E and 1F).

Power supply 102 may include any suitable power supply. For example, power supply 102 may include at least one device and/or a component thereof configured to supply power. Power supply 102 may include a direct current (DC) power supply and/or an alternating current (AC) power supply. Power supply 102 may include at least one of a connection to a power grid, a battery, or any combination thereof. Power supply 102 may convert AC power to DC power. For example, power supply 102 may convert AC power from a power grid to DC power suitable for therapeutic tool 120 and/or power control system 110. For example, power supply 102 may receive AC power from the power grid (e.g., 120 volts AC (VAC) and/or 240 VAC) and convert the AC power to DC power to supply the DC power as output (e.g., 5 amperes (A) at 5.5 volts DC (VDC)) for therapeutic tool 120 and/or power control system 110. Power supply 102 may output power having 5 A of current and/or 5.5 VDC of potential. In other words, the power supply 102 is a non-limiting component of the environment 100.

Power control system 110 may be configured to receive power from power supply 102, to supply power to therapeutic tool 120, or to both receive power from power supply 102 and supply power to therapeutic tool 120, such that power is controlled to prevent, or at least mitigate, excessive tissue damage and/or collateral tissue damage, when the power is used to operate a therapeutic tool 120 involving heating of heating element 122. For example, power control system 110 may be configured to receive power from power supply 102, and supply constant output power to therapeutic tool 120 during a first time interval, and supply pulsed output power to therapeutic tool 120 during a second time interval following the first time interval. In some non-limiting embodiments or aspects, power control system 110 may be a DC power control system. For example, power control system 110 may be configured to receive DC power from power supply 102, and AC power may not be suitable for input to power control system 110. For the purpose of illustration, power control system 110 may not be reasonably connected to an AC or radiofrequency (RF) power supply, because the components of power control system 110 (e.g., first power control circuit 116a, second power control circuit 116b, and/or logic gate(s) 117) may not be functional with AC/RF power as input.

Power control system 110 may include input connection 112, circuit board 114, first power control circuit 116a, second power control circuit 116b, logic gate(s) 117, and/or output connection 118. Non-limiting operative connections of these components are illustrated in FIG. 1B.

Input connection 112 may be configured to receive power from power supply 102. For example, input connection 112 may include any suitable electrical connection to connect to power supply 102 to power control system 110 and/or a component thereof, such as circuit board 114, first power control circuit 116a, and/or second power control circuit 116b. Input connection 112 may include a six-pin connector (e.g., a male or female six-pin connector) or other suitable connector (whether six-pin or not). In some configurations, power control system 110 may be incorporated within power supply 102, and power supply 102 (and/or power control system 110) may be operably connected to provide power as an external power supply to therapeutic tool 120 (e.g., as shown in FIGS. 1E and 1F). In such configurations, input connection 112 may include an internal connection (e.g., within the housing of power supply 102) to power control system 110. For example, input connection 112 may include an internal connection between electrical components of power supply 102 and circuit board 114 and/or the like.

Circuit board 114 may include at least one power control circuit, such as first power control circuit 116a, second power control circuit 116b, and/or logic gate(s) 117, to effect appropriate power control. Circuit board 114 may be configured to receive power from power supply 102 via input connection 112. Circuit board 114 may be configured to supply constant output power during a first time interval to therapeutic tool 120 (via output connection 118), supply pulsed output power to therapeutic tool 120 during a second time interval following the first time interval, and/or stop supplying power to therapeutic tool 120 during a third time interval following the second time interval.

First power control circuit 116a may be configured to supply constant output power during a first time interval, for example, to heat heating element 122 to a target temperature during the first time interval. For example, first power control circuit 116a may be connected to output connection 118 via circuit board 114 and/or configured to supply the constant output power during the first time interval to output connection 118. First power control circuit 116a may be connected to input connection 112 via circuit board 114 and/or configured to receive power therefrom. First power control circuit 116a may include a first one-shot pulse generator circuit (e.g., a monostable multivibrator circuit and/or a monostable pulse generator circuit). The first time interval may include a time interval greater than or equal to 2 seconds and less than or equal to 10 seconds. For example, the first time interval may include a time interval of 2-10 seconds, 2.5-10 seconds, 2-5 seconds, or 2.5-5 seconds. The first time interval may be selected based on a target temperature of heating element 122 of therapeutic tool 120. For example, when tissue is heated in excess of 100° Celsius, the tissue may be broken down and, thus, cut, such as by a cutting element, such as jaws, of therapeutic tool 120. When the tissue is heated to temperatures between 50° to 90° Celsius, the tissue may seal (e.g., weld) to adjacent tissue. For the purpose of illustration, to perform a tissue cutting procedure, the target temperature for heating element 122 may be selected to be greater than or equal to 100° Celsius. To perform a tissue sealing procedure, the target temperature for heating element 122 may be selected to be between 50° and 90° Celsius. To perform a tissue cutting procedure wherein the target tissue is cut and collateral tissue (on both sides of the target tissue) is sealed, the target temperature for heating element 122 may be selected to be greater than or equal to 100° Celsius such that target tissue in contact with and/or within a cut zone around heating element 122 may be cut, while collateral tissue further from heating element 122 (on either side of the cut zone) will only be heated to temperature between 50° and 90° Celsius, causing such collateral tissue to seal at the same time or before the target tissue is cut. Additional details regarding the target temperature for cutting tissue or welding tissue are provided in U.S. Pat. App. Pub. No. 2006/0217706, titled “Tissue Welding and Cutting Apparatus and Method,” the disclosure of which is hereby incorporated by reference herein in its entirety.

A longer first time interval may result in heating of heating element 122 to a higher target temperature because the constant output power heats heating element 122 for a longer amount of time. A shorter first time interval may result in heating of heating element 122 to a lower target temperature because the constant output power heats heating element 122 for a shorter amount of time. Consequently, the first time interval may be selected based on an amount of time needed to heat heating element 122 to the desired target temperature. First power control circuit 116a may be configured based on the selected first time Interval to provide constant output power only during the first time interval.

Heating element 122 may heat at a first rate during the first time interval while first power control circuit 116a supplies constant output power. For example, the first rate at which heating element 122 heats during the first interval may be based on the amplitude of the current of the constant output power, the material properties of heating element 122 and/or components of therapeutic tool 120 proximate heating element 122, the temperature and/or material properties of the environment surrounding heating element 122 (e.g., target tissue, collateral tissue, and/or bodily fluids proximate heating element 122), or any combination thereof.

Second power control circuit 116b may be configured to supply pulsed output power at least during a second time interval following the first time interval, for example, to maintain the temperature of heating element 122 at a relatively constant temperature and/or within a target temperature range during the second time interval. For example, second power control circuit 116b may be connected to output connection 118 via circuit board 114 and/or configured to supply the pulsed output power during the second time interval to output connection 118. Second power control circuit 116b may be connected to input connection 112 via circuit board 114 and/or configured to receive power therefrom. Second power control circuit 116b may include at least one of a second one-shot pulse generator circuit or an oscillator circuit. For example, second power control circuit 116b may include a second one-shot pulse generator circuit and an oscillator circuit. An output of the second one-shot pulse generator circuit may be connected to an input of the oscillator circuit. The second time interval may include a time interval greater than or equal to 5 seconds and less than or equal to 20 seconds. For example, the second time interval may include a time interval of 5-20 seconds, 5-15 seconds, 10-20 seconds, 15-20 seconds, 18 seconds, any multiple of 5-second intervals, or any multiple of 10-second intervals.

The second time interval may be selected based on an average time needed for a procedure or portion thereof during which heat from heating element 122 will be used, such as an average time for a clinician to cut and/or seal a vessel during an EVH procedure, an average time for a clinician to ablate target tissue during an ablation procedure, an average time for a clinician to cut target tissue during a cutting procedure, and/or an average time for a clinician to cauterize tissue during a cauterizing procedure. A longer second time interval may result in a clinician having more time to perform a procedure or portion thereof during which heat from heating element 122 will be used. A shorter second time interval may result in a clinician having less time to perform a procedure or portion thereof during which heat from heating element 122 will be used. Consequently, the second time interval may be selected based on an amount of time during the procedure and/or portion thereof during which heat from heating element 122 will be used. The second one-shot pulse generator circuit of second power control circuit 116b may be configured based on the selected second time interval to provide output to the oscillator circuit only during the second time interval.

The pulsed output power may have a frequency and/or duty cycle selected to maintain a temperature of a heating element 122 of therapeutic tool 120 within a target temperature range. For example, the pulsed output power may have a frequency of greater than or equal to 2.5 Hz and less than or equal to 12 Hz. For example, the pulsed output power may include a frequency of 2.5-12 Hz or 6-12 Hz. The pulsed output power may have a duty cycle of 50%. The pulsed output power may include a periodic square wave, each period of which may include a first duration when the power is low (e.g., off) and a second duration when the power is high (e.g., on). For example, when the pulsed output power is low, heating element 122 may start to cool and there may be a decrease in temperature (within the target temperature range) or temperature may remain substantially constant. When the pulsed output power is high, heating element 122 may be heated thereby causing an increase in temperature (within the target temperature range) or temperature may remain substantially constant by adding heat at the same rate as heat dissipates from the heating element 122. For the purpose of illustration, if the pulsed output power is low at the beginning of the second time interval, heating element 122 may decrease in temperature until the pulsed output power switches to high, and once the pulsed output power is high, heating element 122 may increase in temperature until the pulsed output power switches back to low.

Adjusting the frequency of the pulsed output power may affect the stability of the temperature of heating element 122, such as making the target temperature range wider or narrower. For example, decreasing the frequency will increase the period of the pulsed output power, which may result in relatively longer durations of both low and high power. Consequently, the amount of time heating element 122 is cooling and the amount of time heating element 122 is heating during each period will both be relatively longer, increasing the range of temperatures between the maximum and minimum temperatures of the target temperature range of heating element 122 during the second time interval. Increasing the frequency will decrease the period of the pulsed output power, which may result in relatively shorter durations of both low and high power. Consequently, the amount of time heating element 122 is cooling and the amount of time heating element 122 is heating during each period will both be relatively shorter, decreasing the range of temperatures between the maximum and minimum temperatures of the target temperature range of heating element 122 during the second time interval.

Duty cycle, which may be the ratio of time the pulsed output power is high/on to the total time of the period, may be 50%, resulting in the first duration when the power is low and the second duration when the power is high being equal during each period. If the duty cycle is increased, the amount of time heating element 122 is heating will be relatively longer than the amount of time heating element 122 is cooling. For example, a higher duty cycle may result in an overall increase in temperature of heating element 122 (e.g., over time, over multiple periods, and/or the like), and the overall increase in temperature may still be less than if power simply remained high (e.g., constantly on). If the duty cycle is decreased, the amount of time heating element 122 is heating will be relatively shorter than the amount of time heating element 122 is cooling. For example, a lower duty cycle may result in an overall decrease in temperature of heating element 122 (e.g., over time, over multiple periods, and/or the like), and the overall decrease in temperature may still be less than if power simply remained low (e.g., constantly off).

To maintain the temperature of heating element 122 at a relatively constant temperature within the target temperature range during the second time interval, the frequency and/or duty cycle may be selected so that cooling of heating element 122 during the first duration of low power is offset by heating of heating element 122 during the second duration of high power during each period. To gradually increase the temperature (e.g., increase both the minimum and maximum temperatures of the target temperature range and/or increase the average temperature) of heating element 122 during the second time interval, the frequency and/or duty cycle may be selected so that heating of heating element 122 exceeds cooling of heating element 122 during each period. To gradually decrease the temperature (e.g., decrease both the minimum and maximum temperatures of the target temperature range and/or decrease the average temperature) of heating element 122 during the second time interval, the frequency and/or duty cycle may be selected so that cooling of heating element 122 exceeds heating of heating element 122 during each period.

Heating element 122 may cool at a second rate during durations of each period when the pulsed output power is low, and/or heating element 122 may heat at a third rate during durations of each period when the pulsed output power is high. For example, the second rate at which heating element 122 cools during durations of each period when the pulsed output power is low may be based on the material properties of heating element 122 and/or components of therapeutic tool 120 proximate heating element 122, the temperature and/or material properties of the environment surrounding heating element 122, or any combination thereof. For the purpose of illustration, the second rate at which heating element 122 cools may depend on environmental factors acting as a heat sink. The third rate at which heating element 122 heats during the durations of each period when the pulsed output power is high may be based on the amplitude of the current, the material properties of heating element 122 and/or components of therapeutic tool 120 proximate heating element 122, the temperature and/or material properties of the environment surrounding heating element 122, or any combination thereof. For example, the third rate may be the same as the first rate if the amplitude of the current is the same.

Power control system 110 and/or power supply 102 may include at least one indicator to indicate the first time interval and/or the second time interval. For example, power control system 110 and/or power supply 102 may include an audio indicator, such as a speaker. The audio indicator may produce a first audible indication, such as beeping once per second, during the first time interval. The audio indicator may produce a second audible indication, such as beeping at a frequency faster than once per second, during the second time interval. The audio indicator may provide no audible indication, such as stopping beeping, during the third time interval. Alternatively, in some non-limiting embodiments or aspects, the audio indicator may provide a beeping that is faster during heating than during cooling so a clinician may quickly realize whether the heating element is heating up (i.e., faster beeping) or cooling down (i.e., slower beeping). In some non-limiting embodiments or aspects, a signal line may be connected from power control system 110 to power supply 102, and power supply 102 may sense impedance from power control system 110 based on the signal line. Additionally, power supply 102 may include the audio indicator, and based on the impedance sensed from the signal line, the audio indicator may produce an audible indication (e.g., a beep) intermittently (e.g., once per second) during the first time interval when power control system 110 supplies constant output power to therapeutic tool 120 and may produce audible indications more frequently (e.g., once for every duration of high power during each period) during the second time interval when power control system 110 supplies pulsed output power to therapeutic tool 120.

Power control system 110 may include at least one visual indicator, such as at least one light. For example, the visual indicator may produce a first visual indication, such as blinking once per second and/or illuminating in a first color, during the first time interval. The visual indicator may produce a second visual indication, such as blinking at a frequency faster than once per second and/or illuminating in a second color, during the second time interval. The visual indicator may provide no visual indication, such as stopping blinking and/or illuminating, during the third time interval. Thus, power control system 110 may be configured so there are both audio and visual indicators of heating phase or just one or the other of audio indication and visual indication of heating phase. In some non-limiting embodiments or aspects, the visual indicator may include a red light emitting diode or other red light source that either blinks or remains on during heating and a blue light emitting diode or other blue light source that either blinks or remains on during cooling.

Power control system 110 or circuit board 114 thereof may include at least one logic gate 117. Logic gate(s) 117 may include a first logic gate, and the first logic gate may be connected to receive input from the first power control circuit 116a and the second power control circuit 116b. The first logic gate may be configured to output the constant output power from first power control circuit 116a during the first time interval or the pulsed output power from second power control circuit 116b during the second time interval. For example, the first logic gate may be an OR gate; the output of first power control circuit 116a may be connected to a first input of the OR gate; the output of second power control circuit 116b may be connected to a second input of the OR gate; and the OR gate may supply power from an output thereof when power is supplied at either the first input or second input of the OR gate.

Logic gate(s) 117 may include a second logic gate, and the second logic gate may be connected to the first logic gate and switch 119, which may be in the handle of therapeutic tool 120 and/or on power control system 110. The second logic gate may be configured to output the constant output power or the pulsed output power from the first logic gate only when switch 119 is closed. For example, the second logic gate may be an AND gate; the output of the first logic gate may be connected to a first input of the AND gate; switch 119 may be connected to a second input of the AND gate; and the AND gate may supply power from an output thereof when power is supplied at both the first input and second input of the AND gate. Consequently, when switch 119 is open, power may not flow from power control system 110 to therapeutic tool 120. When switch 119 is closed, constant output power may flow from power control system 110 to therapeutic tool 120 during the first time interval to heat heating element 122 to a target temperature. After the first time interval, pulsed output power may flow from power control system 110 to therapeutic tool 120 during the second time interval to maintain the temperature of heating element 122 at a relatively constant temperature and/or within a target temperature range.

During the third time interval following the second time interval, power control system 110 may not supply power to therapeutic tool 120, for example, which permits cooling of heating element 122 as a way to help avoid or at least mitigate excessive damage to the target tissue, collateral tissue damage, and/or damage to therapeutic tool 120. For example, during the third time interval, first power control circuit 116a and second power control circuit 116b circuit may not supply output power. This cessation of supplying power may prevent supplying power for too long so as to prevent supplying power after a procedure is completed thereby ensuring heating element 122 of therapeutic tool 120 does not overheat and/or heat collateral tissue that is not the target of the procedure.

Heating element 122 may cool at a fourth rate during the third time interval. For example, the fourth rate at which heating element 122 cools during the third time interval may be based on the material properties of heating element 122 and/or components of therapeutic tool 120 proximate heating element 122, the temperature and/or material properties of the environment surrounding heating element 122, or any combination thereof. For the purpose of illustration, the fourth rate may be the same as the second rate, if environmental factors acting as a heat sink are the same.

Opening switch 119 may stop power from flowing from power control system 110 to therapeutic tool 120. In some non-limiting embodiments or aspects, opening switch 119 (e.g., during any of the first time interval, the second time interval, or the third time interval) may reset power control system 110 (e.g., set up power control system 110 for the next procedure, such as endoscopic vessel harvesting procedure). For example, opening switch 119 may cause first power control circuit 116a and/or second power control circuit 116b to reset. This resetting may allow a user to prepare for the next procedure (e.g., endoscopic vessel harvesting procedure), and/or may allow a user to cease heating of heating element 122 of therapeutic tool 120 if a current procedure is completed (e.g., turns out to be completed quickly, such as before the end of the first time interval or before the end of the second time interval). For the purpose of illustration, when switch 119 is closed, constant output power may flow from power control system 110 to therapeutic tool 120 during the first time interval, and pulsed output power may flow from power control system 110 to therapeutic tool 120 during the second time interval, as described herein. During the third time interval following the second time interval, power control system 110 may not supply power to therapeutic tool 120. If switch 119 is opened at any time before the end of the third time interval (e.g., any time during the first, second, or third time interval), power control system 110 may reset such that power is not supplied to therapeutic tool 120 and heating element 122 stops heating. Alternatively, if switch 119 is kept closed through the end of the third time interval and after the end of the third time interval, power control system 110 may still not supply power to therapeutic tool 120 (e.g., until switch 119 is eventually opened so that power control system 110 may reset).

Output connection 118 may be configured to supply power from power control system 110 to therapeutic tool 120. For example, output connection 118 may include any suitable electrical connection to connect power control system 110 to therapeutic tool 120 and/or a component thereof, such as heating element 122 of therapeutic tool 120 that performs one or more therapeutic operations, such as tissue welding, tissue cutting, and/or tissue ablation. Output connection 118 may be connected to first power control circuit 116a and/or second power control circuit 116b via circuit board 114. Output connection 118 may be configured to receive the constant output power from first power control circuit 116a or the pulsed output power from second power control circuit 116b. Output connection 118 may be configured to supply the constant output power or the pulsed output power to therapeutic tool 120. Output connection 118 may include a six-pin connector or other suitable connector (whether six-pin or not).

Power control system 110 may include a housing. For example, the housing may contain input connection 112, circuit board 114, first power control circuit 116a, second power control circuit 116b, logic gate(s) 117, output connection 118, or any combination thereof, or the housing may include multiple sub-housings for housing one or more of these components. For example, the housing may include circuit board 114 (which may include first power control circuit 116a, second power control circuit 116b, and logic gate(s) 117) and output connection 118.

Therapeutic tool 120 may include a tissue ablation device, a tissue cutting device, a tissue cauterizing device, and/or endoscopic vessel harvesting (EVH) device provided with a tissue welding and cutting tool. For example, therapeutic tool 120 may include an EVH device as described in U.S. Pat. No. 9,402,680, titled “Surgical Instrument and Method;” U.S. Pat. No. 7,326,202, titled “Tubular Resistance Heater with Electrically Insulating High Thermal Conductivity Core for Use in a Tissue Welding Device;” or U.S. Pat. No. 7,918,848, titled “Tissue Welding and Cutting Apparatus and Method,” the disclosures of which are hereby incorporated by reference herein in their entireties.

Therapeutic tool 120 may include at least one sensor 124, such as an impedance sensor, capacitance sensor, resistance sensor, pressure sensor, or any combination of such sensors or array of such sensors. For example, sensor(s) 124 may be configured to detect a type of tissue proximate to a cutting element and/or heating element 122 of therapeutic tool 120. At least one of the first time interval, the second time interval, the frequency of the pulsed output power, the duty cycle of the pulsed output power, the amplitude of the current of the constant and/or pulsed output power, or any combination thereof may be selected for the purpose of controlling heating of heating element 122 based on the type of tissue sensed by the (tissue) sensor(s) 124 of therapeutic tool 120. For example, the adjustment of one or more of these characteristics of output power may be based on the target temperature, the target temperature range, and/or the average time for heating of heating element 122 to effect the intended procedure associated with such tissue. For example, the control of heating element 122 of a therapeutic welding and cutting tool of an EVH device may achieve a level of heating sufficient to weld tissue in a first control mode and may achieve a substantially different level of heating to cut tissue in a second control mode, wherein these first and second control modes may be substantially effected by the type of tissue that is undergoing tissue welding and cutting, such as for branch vessels versus a major vein, such as the saphenous vein. By employing appropriate sensor(s) 124, or an array of sensors 124, to provide sensor input to the power control system 110, heating control signals may be tailored to the type of tissue undergoing tissue welding and cutting. For example, in the second control mode, a longer first time interval may result in heating of heating element 122 to a higher temperature needed to cut tissue because the constant output power heats heating element 122 for a longer amount of time. In the first control mode, a shorter first time interval may result in heating of heating element 122 to a lower temperature, such as may be used to weld tissue, because the constant output power heats heating element 122 for a shorter amount of time.

In some non-limiting embodiments or aspects, the first time interval and/or the second time interval may be selected based on a target amount of energy to be supplied to heating element 122 of therapeutic tool 120 during the procedure or portion thereof during which heat from heating element 122 will be used. For example, a polyfuse used in other therapeutic tools may have a nominal resistance of 0.053 ohms and a minimum resistance of 0.034 ohms, and such a polyfuse may have a maximum time to trip of 2.0 seconds when current is 15 A (and resistance is at the minimum, i.e., 0.034 ohms). The power within the polyfuse may be calculated as the product of the square of the current and the resistance (i.e., Power=I²R) to be 7.65 Watts (W), and therefore the energy required to trip the polyfuse may be calculated based on the product of power and time (i.e., Energy=(Power)(time)) to be 15.3 Joules (J). When such a polyfuse is used with 5 A of current, the power through the polyfuse may be calculated based on that current and the nominal resistance (i.e., Power=I²R) to be 1.33 W, and the time to trip the polyfuse may be calculated based on the quotient of energy and power (i.e., time=Energy/Power) to be 11.5 seconds. Given that time, the typical (e.g., average) amount of energy supplied to a heating element of such other therapeutic tools may be calculated based on the current (5 A), the resistance of such a heating element (0.45 ohms), and the aforementioned time (11.5 seconds) to be 129 J, which may be used as the target amount of energy to be supplied to heating element 122 of therapeutic tool 120. In other words, given a constant value for current (e.g., 5 A), the first time interval and/or the second time interval may be selected so that the total amount of energy supplied to heating element 122 during the procedure or portion thereof is approximately 129 J. For example, the first time interval may be selected so that heating element 122 reaches a target temperature, and the second time interval may be selected so that the total energy supplied to heating element 122 during both the first and second time intervals is approximately (e.g., does not exceed) 129 J.

The number and arrangement of systems and/or devices shown in FIGS. 1A-1F are provided as a non-limiting example. Furthermore, two or more systems, devices, or circuits shown in FIGS. 1A-1F may be implemented within a single system, device or circuit, or a single system, device, or circuit shown in FIGS. 1A-1F may be implemented as multiple systems, devices, or circuits.

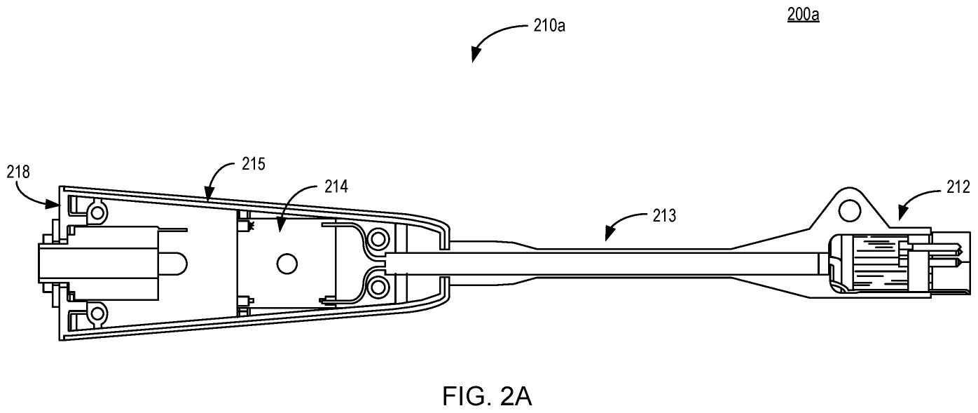

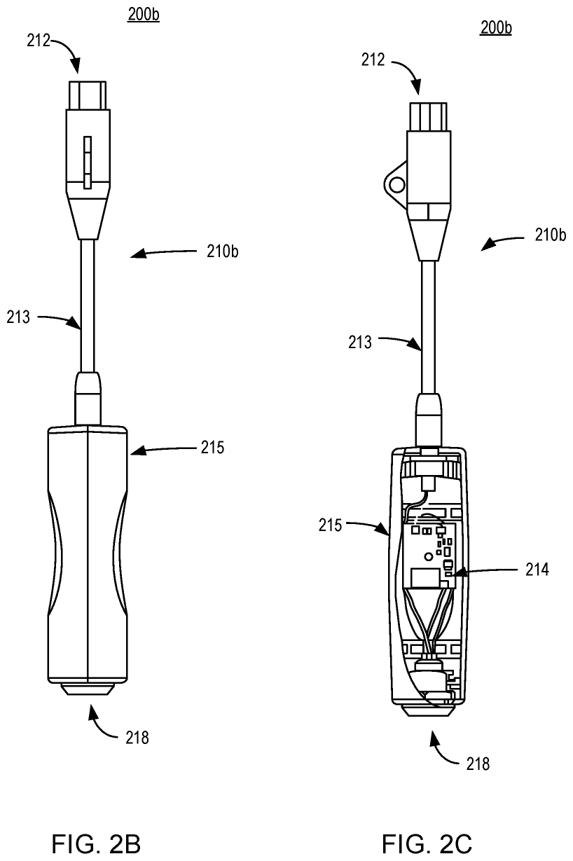

FIG. 2A is a diagram of an exemplary implementation 200a of non-limiting embodiments or aspects relating to a power control system for a therapeutic tool. As shown in FIG. 2A, implementation 200a may include power control system 210a, input connection 212, cable 213, circuit board 214, housing 215, and output connection 218.

Input connection 212 may be connected to a proximal end of cable 213, as described herein. A distal end of cable 213 may be connected to circuit board 214, as described herein. For example, cable 213 may extend from housing 215 to input connection 212, as described herein. Input connection 212 may be configured to receive power from a power supply, such as power supply 102, and/or to supply power via cable 213 to circuit board 214, as described herein.

Circuit board 214 may include at least one power control circuit, as described herein. For example, circuit board 214 may include a first power control circuit, such as first power control circuit 116a. Circuit board 214 may include a second power control circuit, such as second power control circuit 116b. Circuit board 214 may be configured to supply constant output power during a first time interval, as described herein. Circuit board 214 may be configured to supply pulsed output power during a second time interval following the first time interval, as described herein. Circuit board 214 may include at least one logic gate, such as logic gate(s) 117. Output connection 218 may receive the constant output power or the pulsed output power from circuit board 214 and supply the output power to a therapeutic tool, as described herein.

FIGS. 2B and 2C are diagrams of an exemplary implementation 200b of non-limiting embodiments or aspects relating to a power control system for a therapeutic tool. As shown in FIGS. 2B and 2C, implementation 200b may include power control system 210b, input connection 212, cable 213, circuit board 214, housing 215, and output connection 218.

Input connection 212 may be connected to a proximal end of cable 213, as described herein. A distal end of cable 213 may be connected to circuit board 214, as described herein. For example, cable 213 may extend from housing 215 to input connection 212, as described herein. Input connection 212 may be configured to receive power from a power supply, such as power supply 102, and/or to supply power via cable 213 to circuit board 214, as described herein.

Circuit board 214 may include at least one power control circuit, as described herein. For example, circuit board 214 may include a first power control circuit and a second power control circuit, such as first power control circuit 116a and second power control circuit 116b, respectively. Circuit board 214 may be configured to supply constant output power during a first time interval and to supply pulsed output power during a second time interval following the first time interval, as described herein. Circuit board 214 may include at least one logic gate, such as logic gate(s) 117. Output connection 218 may receive the constant output power or the pulsed output power from circuit board 214 and supply the output power to a therapeutic tool, as described herein.

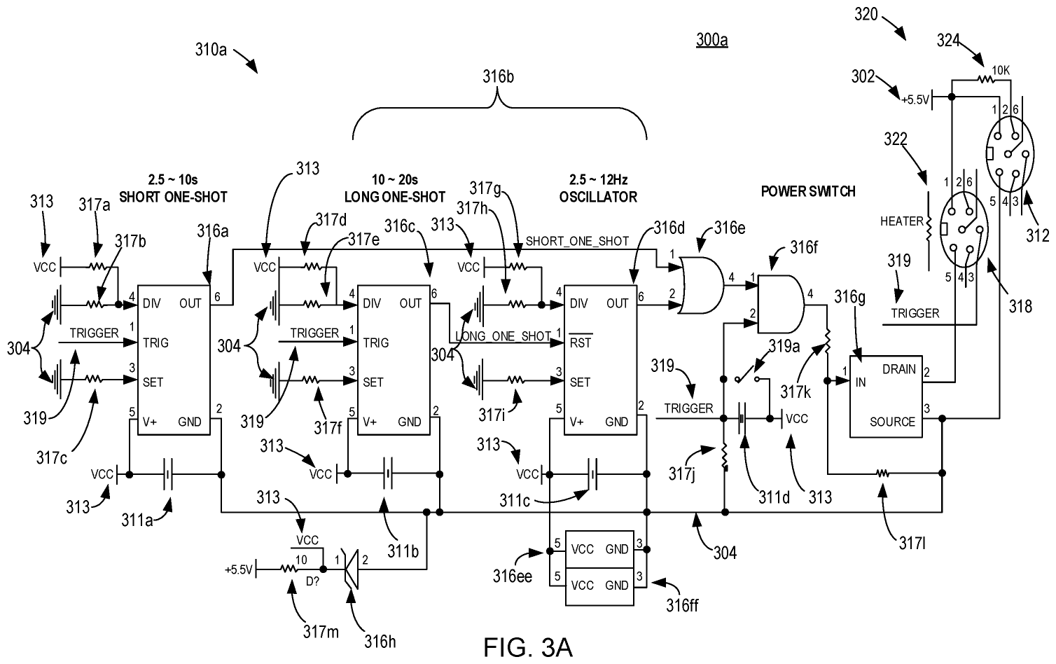

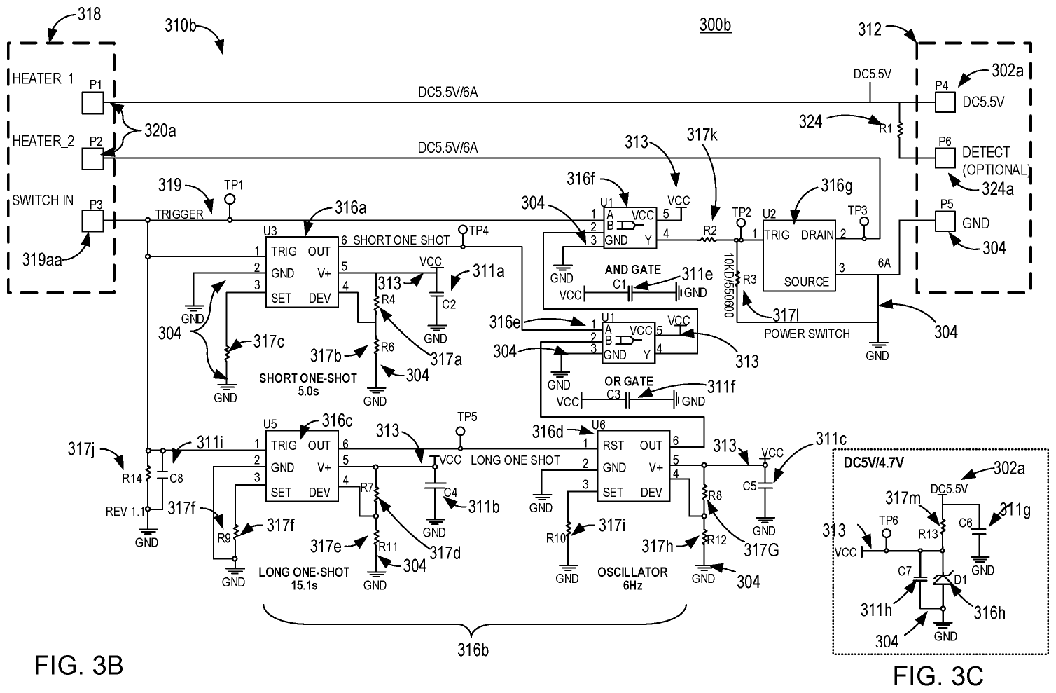

FIG. 3A is a circuit diagram of an exemplary implementation 300a of non-limiting embodiments or aspects relating to a variable power modulation profile power control system for a therapeutic tool. As shown in FIG. 3A, implementation 300a may include power supply 302, ground 304, power control system 310a, capacitors 311a-311d, input connection 312, input power line 313, first power control circuit 316a, second power control circuit 316b, second one-shot pulse generator circuit 316c, oscillator circuit 316d, first logic gate 316e, second logic gate 316f, power switch 316g, Zener diode 316h, resistors 317a-317m, output connection 318, trigger signal line 319, trigger switch 319a, therapeutic tool 320, heating element 322, and/or feedback resistor 324.

Input connection 312 may be configured to receive power from power supply 302, as described herein. For example, input connection 312 may include any suitable electrical connection to connect to power supply 302 to power control system 310a. For the purpose of illustration, input connection 312 may include a six-pin connector.

Input power line 313 may be configured to receive power from input connection 312. For example, input power line 313 may include any suitable electrical connection to connect input connection 312 to first power control circuit 316a and/or second power control circuit 316b (e.g., second one-shot pulse generator circuit 316c and oscillator circuit 316d). For the purpose of illustration, resistor 317m may be connected between an input voltage (e.g., 5.5 V) from input connection 312 and input power line 313, and Zener diode 316h may be connected between input power line 313 and ground 304. As such, Zener diode 316h and resistor 317m may form a regulator to stabilize the voltage on input power line 313.

Power control system 310a may be configured to receive power from power supply 302 via input power line 313, to supply constant output power during a first time interval, and/or to supply pulsed output power during a second time interval following the first time interval, as described herein. For example, first power control circuit 316a may be connected to input power line 313 and/or may be configured to supply constant output power during the first time interval to heat heating element 122 to a target temperature during the first time interval, as described herein. For example, first power control circuit 316a may be connected (directly or indirectly) to output connection 318 and/or configured to supply the constant output power during the first time interval to output connection 318. First power control circuit 316a may be connected to input power line 313. First power control circuit 316a may include a first one-shot pulse generator circuit. The first time interval may be selected based on a target temperature of heating element 322 of therapeutic tool 320, as described herein.

Second power control circuit 316b may include second one-shot pulse generator circuit 316c and oscillator circuit 316d, which may be connected to input power line 313 and/or may be configured to supply pulsed output power at least during the second time interval following the first time interval, for example, to maintain the temperature of heating element 322 at a relatively constant temperature within a target temperature range during the second time interval, as described herein. For example, second power control circuit 316b may be connected (directly or indirectly) to output connection 318 and/or configured to supply the pulsed output power during the second time interval to output connection 318. Second power control circuit 316b may be connected to input power line 313. An output of second one-shot pulse generator circuit 316c may be connected to an input of oscillator circuit 316d. The second time interval may be selected based on an average time for a procedure or portion thereof during which heat from heating element 322 will be used, as described herein. Second one-shot pulse generator circuit 316c may be configured based on the selected second time interval to provide output to oscillator circuit 316d only during the second time interval or only during the first time interval and the second time interval. The pulsed output power (from oscillator circuit 316d) may have a frequency and/or duty cycle selected to maintain a temperature of a heating element 322 of therapeutic tool 320 within a target temperature range, as described herein

First power control circuit 316a and/or second power control circuit 316b may be connected via first logic gate 316e and second logic gate 316f to output connection 318, as described herein. For example, first logic gate 316e may be connected to first power control circuit 316a and second power control circuit 316b. First logic gate 316e may be configured to output the constant output power from first power control circuit 316a during the first time interval or the pulsed output power from second power control circuit 316b during the second time interval. For example, first logic gate 316e may be an OR gate, as described herein. Second logic gate 316f may be connected to first logic gate 316e and trigger switch 319a via trigger signal line 319. Second logic gate 316f may be configured to output the constant output power or the pulsed output power from first logic gate 316e only when trigger switch 319a is closed, as described herein. For example, second logic gate 316f may be an AND gate, as described herein. Consequently, when trigger switch 319a is open, power may not flow from power control system 310a to therapeutic tool 320. When trigger switch 319a is closed, constant output power may flow from power control system 310a to therapeutic tool 320 during the first time interval, and pulsed output power may flow from power control system 310a to therapeutic tool 320 during the second time interval, as described herein.

Output connection 318 may be configured to receive the constant output power from first power control circuit 316a or the pulsed output power from second power control circuit 316b. Output connection 318 may be configured to supply the constant output power or the pulsed output power to therapeutic tool 320 and/or heating element 322, as described herein.

First power control circuit 316a may include a first one-shot pulse generator circuit. For example, first power control circuit 316a may include an Analog Devices LTC6993IS6-2 TimerBlox: Monostable Pulse Generator (One Shot). First power control circuit 316a may include a plurality of inputs, such as input pins. For example, first power control circuit 316a may include a supply voltage input (V+), a programmable divider and polarity input (DIV), a pulse width setting input (SET), and/or a trigger input (TRIG). First power control circuit 316a may include at least one output (OUT). First power control circuit 316a may include a ground connection (GND). Input power line 313 may be connected to the supply voltage input (V+) of first power control circuit 316a, and/or ground 304 may be connected to the ground connection (GND) of first power control circuit 316a. Capacitor 311a may be connected between supply voltage input (V+) and ground connection (GND) of first power control circuit 316a.

The programmable divider and polarity input (DIV) of first power control circuit 316a may be connected between resistor 317a and resistor 317b. Input power line 313 may be connected to resistor 317a on an opposite side of resistor 317a from the programmable divider and polarity input (DIV). Ground 304 may be connected to resistor 317b on an opposite side of resistor 317b from the programmable divider and polarity input (DIV). As such, resistor 317a and resistor 317b may form a voltage divider to set an internal clock divider based on the values of these resistors. For example, resistor 317a may have a resistance value of 1000 kiloohms (kΩ) and resistor 317b may have a resistance value of 887 kΩ, and this may result in the internal clock divider being set to a value of 2,097,152, which may be suitable for a first time interval between 2.097 seconds and 33.55 seconds. A trigger input (TRIG) of first power control circuit 316a may be connected to trigger signal line 319. A pulse width setting input (SET) of first power control circuit 316a may be connected to resistor 317c. Ground 304 may be connected to resistor 317c on an opposite side of resistor 317c from the pulse width setting input (SET). As such, the first time interval (i.e., the output pulse width of first power control circuit 316a) may be set based on the value of resistor 317c and the internal clock divider. In some non-limiting embodiments or aspects, resistor 317c may be a variable resistor with a resistance value that is variable, and the first time interval may be set (within the range of 2.097 seconds and 33.55 seconds determined by the internal clock divider) based on a selected value of the resistance of resistor 317c. An output (OUT) of first power control circuit 316a may be connected to a first input of first logic gate 316e.

Second power control circuit 316b may include second one-shot pulse generator circuit 316c and oscillator circuit 316d. For example, second one-shot pulse generator circuit 316c may include an Analog Devices LTC6993IS6-2 TimerBlox: Monostable Pulse Generator (One Shot). For example, oscillator circuit 316d may include an Analog Devices LTC6995IS6-2 TimerBlox: Long Timer, Low Frequency Oscillator.

Second one-shot pulse generator circuit 316c may include a plurality of inputs. For example, second one-shot pulse generator circuit 316c may include a supply voltage input (V+), a programmable divider and polarity input (DIV), a pulse width setting input (SET), and/or a trigger input (TRIG). Second one-shot pulse generator circuit 316c may include at least one output (OUT). Second one-shot pulse generator circuit 316c may include a ground connection (GND). Input power line 313 may be connected to the supply voltage input (V+) of second one-shot pulse generator circuit 316c. Ground 304 may be connected to the ground connection (GND) of second one-shot pulse generator circuit 316c. Capacitor 311b may be connected between supply voltage input (V+) and ground connection (GND) of second one-shot pulse generator circuit 316c.

The programmable divider and polarity input (DIV) of second one-shot pulse generator circuit 316c may be connected between resistor 317d and resistor 317e. Input power line 313 may be connected to resistor 317d on an opposite side of resistor 317d from the programmable divider and polarity input (DIV). Ground 304 may be connected to resistor 317e on an opposite side of resistor 317e from the programmable divider and polarity input (DIV). As such, resistor 317d and resistor 317e may form a voltage divider to set an internal clock divider based on the values of these resistors. For example, resistor 317d may have a resistance value of 1000 kΩ and resistor 317e may have a resistance value of 887 kΩ, and this may result in the internal clock divider being set to a value of 2,097,152, which may be suitable for a second time interval between 2.097 seconds and 33.55 seconds. A trigger input (TRIG) of second one-shot pulse generator circuit 316c may be connected to trigger signal line 319. A pulse width setting input (SET) of second one-shot pulse generator circuit 316c may be connected to resistor 317f. Ground 304 may be connected to resistor 317f on an opposite side of resistor 317f from the pulse width setting input (SET). As such, the second time interval (i.e., the output pulse width of second one-shot pulse generator circuit 316c) may be set based on the value of resistor 317f and the internal clock divider. In some non-limiting embodiments or aspects, resistor 317f may be a variable resistor with a resistance value that is variable, and the second time interval may be set (within the range of 2.097 seconds and 33.55 seconds determined by the internal clock divider) based on a selected value of the resistance of resistor 317f. An output (OUT) of second one-shot pulse generator circuit 316c may be connected to a reset input (RST) of oscillator circuit 316d.

Oscillator circuit 316d may include a plurality of inputs. For example, oscillator circuit 316d may include a supply voltage input (V+), a programmable divider and polarity input (DIV), a frequency-setting input (SET), and/or a reset input (RST). Oscillator circuit 316d may include at least one output (OUT). Oscillator circuit 316d may include a ground connection (GND). Input power line 313 may be connected to the supply voltage input (V+) of oscillator circuit 316d. Ground 304 may be connected to the ground connection (GND) of oscillator circuit 316d. Capacitor 311c may be connected between supply voltage input (V+) and ground connection (GND) of oscillator circuit 316d.

The programmable divider and polarity input (DIV) of oscillator circuit 316d may be connected between resistor 317g and resistor 317h. Input power line 313 may be connected to resistor 317g on an opposite side of resistor 317g from the programmable divider and polarity input (DIV). Ground 304 may be connected to resistor 317h on an opposite side of resistor 317h from the programmable divider and polarity input (DIV). As such, resistor 317g and resistor 317h may form a voltage divider to set an internal clock divider based on the values of these resistors. For example, resistor 317g may have a resistance value of 976 kΩ and resistor 317h may have a resistance value of 182 kΩ, and this may result in the internal clock divider being set to a value of 64, which may be suitable for a period of 65.5 milliseconds (ms) to 1.05 seconds, wherein the frequency (f) is the reciprocal of the period (P) (i.e., f=1/P). A reset input (RST) of oscillator circuit 316d may be connected to the output of second one-shot pulse generator circuit 316c. A frequency-setting input (SET) of oscillator circuit 316d may be connected to resistor 317i. Ground 304 may be connected to resistor 317i on an opposite side of resistor 317i from the pulse width setting input (SET). As such, the frequency of the pulsed output power (i.e., the reciprocal of the period of the periodic output of oscillator circuit 316d) may be set based on the value of resistor 317i and the internal clock divider. In some non-limiting embodiments or aspects, resistor 317i may be a variable resistor with a resistance value that is variable, and the frequency may be set based on a selected value of the resistance of resistor 317i. An output (OUT) of oscillator circuit 316d may be connected to a second input of first logic gate 316e.

First logic gate 316e may be connected to first power control circuit 316a and oscillator circuit 316d of second power control circuit 316b. For example, the output of first power control circuit 316a may be connected to a first input of first logic gate 316e, and the output of oscillator circuit 316d may be connected to a second input of first logic gate 316e. First logic gate 316e may be configured to output the constant output power from first power control circuit 316a during the first time interval or the pulsed output power from oscillator circuit 316d during the second time interval, as described herein. First logic gate 316e may be an OR gate, as described herein. First logic gate 316e may include power supply pins 316ee (e.g., including a supply voltage input (VCC) and/or a ground connection (GND)).

Second logic gate 316f may be connected to first logic gate 316e and trigger switch 319a. For example, an output of first logic gate 316e may be connected to a first input of second logic gate 316f, and the trigger signal line 319, which is connected to trigger switch 319a, may be connected to a second input of second logic gate 316f. Second logic gate 316f may be configured to output the output of first logic gate 316e only when trigger switch 319a is closed, as described herein. For example, trigger switch 319a may be open by default, and may be closed by a user when performing a procedure with therapeutic tool 320. Second logic gate 316f may be an AND gate, as described herein. Second logic gate 316f may include power supply pins 316ff (e.g., including a supply voltage input (VCC) and/or a ground connection (GND)).

Trigger switch 319a may be in the handle of power control system 310a or therapeutic tool 320. Capacitor 311d may be connected between input power line 313 and trigger signal line 319. Resistor 317j may be connected between trigger signal line 319 and ground 304.

The output of second logic gate 316f may be connected to power switch 316g. Resistor 317k may be connected between the output of second logic gate 316f and power switch 316g. Power switch 316g may include an Infineon Smart Low Side Power Switch Power HITFET BTS 134D. Power switch 316g may include at least one input. For example, power switch 316g may include an input (IN), which may be connected to a gate electrode of a transistor, such as an N-channel field-effect transistor (FET). Power switch 316g may include at least one ground connection (SOURCE), which may be connected to a source electrode of the transistor. Power switch 316g may include at least one output (DRAIN), which may be connected to a drain electrode of the transistor. Resistor 317l may be connected between the input (IN) and the ground connection (SOURCE) of power switch 316g. Power switch 316g may receive the output power from second logic gate 316f and provide that output power on the output (DRAIN) of power switch 316g.

The output (DRAIN) of power switch 316g may be connected to at least one of output connection 318. For example, the output (DRAIN) of power switch 316g may be connected to a pin (e.g., pin 5) of output connection 318. The ground connection (SOURCE) of power switch 316g may be connected to at least one of ground 304 and/or input connection 312. For example, the ground connection (SOURCE) of power switch 316g may be connected to a pin (e.g., pin 5) of input connection 312. Trigger signal line 319 may be connected to output connection 318. For example, trigger signal line 319 may be connected to a pin (e.g., pin 3) of output connection 318.

Therapeutic tool 320 may include heating element 322, as described herein. For example, heating element 322 may include a resistive heating element, as described herein. Heating element 322 may be configured to receive the constant output power or the pulsed output power from power control system 310a and/or may heat based on the received power. Heating element 322 may increase in temperature during the first time interval based on the constant output power received at heating element 322 until reaching a target temperature, as described herein. During the second time interval, a temperature of heating element 322 may be maintained within a target temperature range based on the pulsed output power received at heating element 322, as described herein. For example, when the pulsed output power is high, heating element 322 may increase in temperature within the target temperature range, and/or when the pulsed output power is low, heating element 322 may decrease in temperature within the target temperature range.

Feedback resistor 324 may be connected to at least one of output connection 318 and/or input connection 312. For example, one side of feedback resistor 324 may be connected to a pin (e.g., pin 1) of both output connection 318 and input connection 312, and a second side of feedback resistor 324 may be connected to a pin (e.g., pin 2) of input connection 312. Power supply 302 may receive a signal based on the current through feedback resistor 324. Power supply 302 may turn on based on receiving the signal from feedback resistor 324. Feedback resistor 324 may be a 10 kΩ resistor.

For the purpose of illustration, upon the closing of trigger switch 319a, power is supplied from power supply 302 to input connection 312, which supplies power to input power line 313. Input power line 313 supplies power to first power control circuit 316a and second power control circuit 316b (i.e., to second one-shot pulse generator circuit 316c and oscillator circuit 316d). First power control circuit 316a supplies constant output power during the first time interval, which is set based on the resistors 317a, 317b, and 317c. The output power from first power control circuit 316a is supplied to first logic gate 316e, which in turn supplies the constant output power to second logic gate 316f, and, since trigger switch 319a is closed, second logic gate 316f, supplies the constant output power to output connection 318. Output connection 318 supplies the constant output power to therapeutic tool 320, which supplies the constant output power to heat heating element 322 to a target temperature during the first time interval. After the first time interval, first power control circuit 316a stops supplying the constant output power.

During the second time interval following the first time interval (or during an entire time interval including both the first and second time interval), second one-shot pulse generator circuit 316c supplies constant output power to oscillator circuit 316d. The (second or entire) time interval during which second one-shot pulse generator circuit 316c supplies constant output power is set based on the resistors 317d, 317e, and 317f. Oscillator circuit 316d supplies pulsed output power for as long as second one-shot pulse generator circuit 316c supplies constant output power. The frequency of the pulsed output power from oscillator circuit 316d is set based on resistors 317g, 317h, and 317i. The output power from oscillator circuit 316d is supplied to first logic gate 316e, which in turn supplies the pulsed output power to second logic gate 316f during the second time interval (since the constant output power from first power control circuit 316a stopped after the first time interval). Since trigger switch 319a is closed, second logic gate 316f, supplies the pulsed output power to output connection 318. Output connection 318 supplies the pulsed output power to therapeutic tool 320, which supplies the pulsed output power to heating element 322 to maintain the temperature of heating element 322 at a relatively constant temperature and/or within a target temperature range during the second time interval. After the second time interval, second one-shot pulse generator circuit 316c stops supplying power to oscillator circuit 316d, which, therefore, stops supplying the pulsed output power.

FIGS. 3B and 3C are circuit diagrams of an exemplary implementation 300b of non-limiting embodiments or aspects relating to a fixed power modulation profile power control system for a therapeutic tool. As shown in FIGS. 3B and 3C, implementation 300b may include power supply input 302a, ground 304, power control system 310b, capacitors 311a-311c and 311e-311i, input connection 312, input power line 313, first power control circuit 316a, second power control circuit 316b, second one-shot pulse generator circuit 316c, oscillator circuit 316d, first logic gate 316e, second logic gate 316f, power switch 316g, Zener diode 316h, resistors 317a-317m, output connection 318, trigger signal line 319, trigger switch input 319aa, therapeutic tool/heating element outputs 320a, feedback resistor 324, and/or feedback output 324a.

Input connection 312 may be configured to receive power from power supply input 302a, as described herein. For example, input connection 312 may include any suitable electrical connection to connect to power supply input 302a to power control system 310b. For the purpose of illustration, input connection 312 may include a six-pin connector.

Input power line 313 may be configured to receive power from input connection 312. For example, input power line 313 may include any suitable electrical connection to connect input connection 312 to first power control circuit 316a and/or second power control circuit 316b (e.g., second one-shot pulse generator circuit 316c and oscillator circuit 316d). For the purpose of illustration, as shown in FIG. 3C, resistor 317m may be connected between power supply input 302a (e.g., 5.5 V) from input connection 312 and input power line 313, and Zener diode 316h may be connected between input power line 313 and ground 304. As such, Zener diode 316h and resistor 317m may form a regulator to stabilize the voltage on input power line 313. Capacitor 311g may be connected between power supply input 302a and ground 304, and capacitor 311h may be connected between input power line 313 and ground 304.

Power control system 310b may be configured to receive power from power supply input 302a via input power line 313 to supply constant output power during a first time interval, and/or to supply pulsed output power during a second time interval following the first time interval, as described herein. For example, first power control circuit 316a may be connected to input power line 313 and/or may be configured to supply constant output power during the first time interval to heat a heating element connected to therapeutic tool/heating element outputs 320a to a target temperature during the first time interval, as described herein. For example, first power control circuit 316a may be connected (directly or indirectly) to output connection 318 and/or configured to supply the constant output power during the first time interval to output connection 318. First power control circuit 316a may include a first one-shot pulse generator circuit. The first time interval may be selected based on a target temperature of the heating element, as described herein.

Second power control circuit 316b may include second one-shot pulse generator circuit 316c and oscillator circuit 316d, which may be connected to input power line 313 and/or may be configured to supply pulsed output power at least during the second time interval following the first time interval, for example, to maintain the temperature of the heating element connected to therapeutic tool/heating element outputs 320a at a relatively constant temperature within a target temperature range during the second time interval, as described herein. For example, second power control circuit 316b may be connected (directly or indirectly) to output connection 318 and/or configured to supply the pulsed output power during the second time interval to output connection 318. An output of second one-shot pulse generator circuit 316c may be connected to an input of oscillator circuit 316d. The second time interval may be selected based on an average time for a procedure or portion thereof during which heat from the heating element will be used, as described herein. Second one-shot pulse generator circuit 316c may be configured based on the selected second time interval to provide output to oscillator circuit 316d only during the second time interval or only during the first time interval and the second time interval. The pulsed output power (from oscillator circuit 316d) may have a frequency and/or duty cycle selected to maintain a temperature of the heating element within a target temperature range, as described herein

First power control circuit 316a and/or second power control circuit 316b may be connected via first logic gate 316e and second logic gate 316f to output connection 318, as described herein. For example, first logic gate 316e may be connected to first power control circuit 316a and second power control circuit 316b. First logic gate 316e may be configured to output the constant output power from first power control circuit 316a during the first time interval or the pulsed output power from second power control circuit 316b during the second time interval. For example, first logic gate 316e may be an OR gate, as described herein. Second logic gate 316f may be connected to first logic gate 316e and trigger switch input 319aa via trigger signal line 319. Second logic gate 316f may be configured to output the constant output power or the pulsed output power from first logic gate 316e only when trigger switch input 319aa is high (corresponding to a trigger switch being closed), as described herein. For example, second logic gate 316f may be an AND gate, as described herein. Consequently, when trigger switch input 319aa is low (open switch), power may not flow from power control system 310b to therapeutic tool/heating element outputs 320a. When trigger switch input 319aa is high (closed switch), constant output power may flow from power control system 310b to therapeutic tool/heating element outputs 320a during the first time interval, and pulsed output power may flow from power control system 310b to therapeutic tool/heating element outputs 320a during the second time interval, as described herein.

Output connection 318 may be configured to receive the constant output power from first power control circuit 316a or the pulsed output power from second power control circuit 316b. Output connection 318 may be configured to supply the constant output power or the pulsed output power to a therapeutic tool and/or heating element thereof via therapeutic tool/heating element outputs 320a, as described herein.

First power control circuit 316a may include a first one-shot pulse generator circuit. For example, first power control circuit 316a may include an Analog Devices LTC6993IS6-2 TimerBlox: Monostable Pulse Generator (One Shot). First power control circuit 316a may include a plurality of inputs, such as input pins. For example, first power control circuit 316a may include a supply voltage input (V+), a programmable divider and polarity input (DIV), a pulse width setting input (SET), and/or a trigger input (TRIG). First power control circuit 316a may include at least one output (OUT). First power control circuit 316a may include a ground connection (GND). Input power line 313 may be connected to the supply voltage input (V+) of first power control circuit 316a, and/or ground 304 may be connected to the ground connection (GND) of first power control circuit 316a. Capacitor 311a may be connected between supply voltage input (V+) and ground connection (GND) of first power control circuit 316a.

The programmable divider and polarity input (DIV) of first power control circuit 316a may be connected between resistor 317a and resistor 317b. Input power line 313 may be connected to resistor 317a on an opposite side of resistor 317a from the programmable divider and polarity input (DIV). Ground 304 may be connected to resistor 317b on an opposite side of resistor 317b from the programmable divider and polarity input (DIV). As such, resistor 317a and resistor 317b may form a voltage divider to set an internal clock divider based on the values of these resistors. For example, resistor 317a may have a resistance value of 1000 kΩ and resistor 317b may have a resistance value of 887 kΩ, and this may result in the internal clock divider being set to a value of 2,097,152, which may be suitable for a first time interval between 2.097 seconds and 33.55 seconds. A trigger input (TRIG) of first power control circuit 316a may be connected to trigger signal line 319. A pulse width setting input (SET) of first power control circuit 316a may be connected to resistor 317c. Ground 304 may be connected to resistor 317c on an opposite side of resistor 317c from the pulse width setting input (SET). As such, the first time interval (i.e., the output pulse width of first power control circuit 316a) may be set based on the value of resistor 317c and the internal clock divider. In some non-limiting embodiments or aspects, resistor 317c may have a resistance value of 120 kΩ, and the first time interval may therefore be set at 5.0 seconds. An output (OUT) of first power control circuit 316a may be connected to a first input of first logic gate 316e.

Second power control circuit 316b may include second one-shot pulse generator circuit 316c and oscillator circuit 316d. For example, second one-shot pulse generator circuit 316c may include an Analog Devices LTC6993IS6-2 TimerBlox: Monostable Pulse Generator (One Shot). For example, oscillator circuit 316d may include an Analog Devices LTC6995IS6-2 TimerBlox: Long Timer, Low Frequency Oscillator.

Second one-shot pulse generator circuit 316c may include a plurality of inputs. For example, second one-shot pulse generator circuit 316c may include a supply voltage input (V+), a programmable divider and polarity input (DIV), a pulse width setting input (SET), and/or a trigger input (TRIG). Second one-shot pulse generator circuit 316c may include at least one output (OUT). Second one-shot pulse generator circuit 316c may include a ground connection (GND). Input power line 313 may be connected to the supply voltage input (V+) of second one-shot pulse generator circuit 316c. Ground 304 may be connected to the ground connection (GND) of second one-shot pulse generator circuit 316c. Capacitor 311b may be connected between supply voltage input (V+) and ground connection (GND) of second one-shot pulse generator circuit 316c.

The programmable divider and polarity input (DIV) of second one-shot pulse generator circuit 316c may be connected between resistor 317d and resistor 317e. Input power line 313 may be connected to resistor 317d on an opposite side of resistor 317d from the programmable divider and polarity input (DIV). Ground 304 may be connected to resistor 317e on an opposite side of resistor 317e from the programmable divider and polarity input (DIV). As such, resistor 317d and resistor 317e may form a voltage divider to set an internal clock divider based on the values of these resistors. For example, resistor 317d may have a resistance value of 1000 kΩ and resistor 317e may have a resistance value of 887 kΩ, and this may result in the internal clock divider being set to a value of 2,097,152, which may be suitable for a second time interval between 2.097 seconds and 33.55 seconds. A trigger input (TRIG) of second one-shot pulse generator circuit 316c may be connected to trigger signal line 319. A pulse width setting input (SET) of second one-shot pulse generator circuit 316c may be connected to resistor 317f. Ground 304 may be connected to resistor 317f on an opposite side of resistor 317f from the pulse width setting input (SET). As such, the second time interval (i.e., the output pulse width of second one-shot pulse generator circuit 316c) may be set based on the value of resistor 317f and the internal clock divider. In some non-limiting embodiments or aspects, resistor 317f may have a resistance value of 360 kΩ, and the second time interval may therefore be set at 15.1 seconds. An output (OUT) of second one-shot pulse generator circuit 316c may be connected to a reset input (RST) of oscillator circuit 316d.

Oscillator circuit 316d may include a plurality of inputs. For example, oscillator circuit 316d may include a supply voltage input (V+), a programmable divider and polarity input (DIV), a frequency-setting input (SET), and/or a reset input (RST). Oscillator circuit 316d may include at least one output (OUT). Oscillator circuit 316d may include a ground connection (GND). Input power line 313 may be connected to the supply voltage input (V+) of oscillator circuit 316d. Ground 304 may be connected to the ground connection (GND) of oscillator circuit 316d. Capacitor 311c may be connected between supply voltage input (V+) and ground connection (GND) of oscillator circuit 316d.

The programmable divider and polarity input (DIV) of oscillator circuit 316d may be connected between resistor 317g and resistor 317h. Input power line 313 may be connected to resistor 317g on an opposite side of resistor 317g from the programmable divider and polarity input (DIV). Ground 304 may be connected to resistor 317h on an opposite side of resistor 317h from the programmable divider and polarity input (DIV). As such, resistor 317g and resistor 317h may form a voltage divider to set an internal clock divider based on the values of these resistors. For example, resistor 317g may have a resistance value of 976 kΩ and resistor 317h may have a resistance value of 182 kΩ, and this may result in the internal clock divider being set to a value of 64, which may be suitable for a period of 65.5 milliseconds (ms) to 1.05 seconds, wherein the frequency (f) is the reciprocal of the period (P) (i.e., f=1/P). A reset input (RST) of oscillator circuit 316d may be connected to the output of second one-shot pulse generator circuit 316c. A frequency-setting input (SET) of oscillator circuit 316d may be connected to resistor 317i. Ground 304 may be connected to resistor 317i on an opposite side of resistor 317i from the pulse width setting input (SET). As such, the frequency of the pulsed output power (i.e., the reciprocal of the period of the periodic output of oscillator circuit 316d) may be set based on the value of resistor 317i and the internal clock divider. In some non-limiting embodiments or aspects, resistor 317i may have a resistance value of 127 kΩ, and the period of the output may therefore be set at 166.5 ms (corresponding to about 6 Hz). An output (OUT) of oscillator circuit 316d may be connected to a second input of first logic gate 316e.

First logic gate 316e may be connected to first power control circuit 316a and oscillator circuit 316d of second power control circuit 316b. For example, the output of first power control circuit 316a may be connected to a first input of first logic gate 316e, and the output of oscillator circuit 316d may be connected to a second input of first logic gate 316e. First logic gate 316e may be configured to output the constant output power from first power control circuit 316a during the first time interval or the pulsed output power from oscillator circuit 316d during the second time interval, as described herein. First logic gate 316e may be an OR gate, as described herein. First logic gate 316e may be connected to input power line 313 and ground 304, and capacitor 311e may be connected between input power line 313 and ground 304 across first logic gate 316e.

Second logic gate 316f may be connected to first logic gate 316e and trigger switch 319a. For example, an output of first logic gate 316e may be connected to a first input of second logic gate 316f, and the trigger signal line 319, which is connected to trigger switch 319a, may be connected to a second input of second logic gate 316f. Second logic gate 316f may be configured to output the output of first logic gate 316e only when trigger switch 319a is closed, as described herein. For example, trigger switch 319a may be open by default, and may be closed by a user when performing a procedure with therapeutic tool 320. Second logic gate 316f may be an AND gate, as described herein. Second logic gate 316f may be connected to input power line 313 and ground 304, and capacitor 311f may be connected between input power line 313 and ground 304 across first logic gate 316e.

A trigger switch may be in the handle of power control system 310b or a therapeutic tool and may be connected to trigger switch input 319aa. Capacitor 311i may be connected between trigger signal line 319 and ground 304, and resistor 317j may be connected between trigger signal line 319 and ground 304.

The output of second logic gate 316f may be connected to power switch 316g. Resistor 317k may be connected between the output of second logic gate 316f and power switch 316g. Power switch 316g may include an Infineon Smart Low Side Power Switch Power HITFET BTS 134D. Power switch 316g may include at least one input. For example, power switch 316g may include an input (TRIG), which may be connected to a gate electrode of a transistor, such as an N-channel field-effect transistor (FET). Power switch 316g may include at least one ground connection (SOURCE), which may be connected to a source electrode of the transistor. Power switch 316g may include at least one output (DRAIN), which may be connected to a drain electrode of the transistor. Resistor 317l may be connected between the input (TRIG) and the ground connection (SOURCE) of power switch 316g. Power switch 316g may receive the output power from second logic gate 316f and provide that output power on the output (DRAIN) of power switch 316g.

The output (DRAIN) of power switch 316g may be connected to at least one of output connection 318. For example, the output (DRAIN) of power switch 316g may be connected to therapeutic tool/heating element outputs 320a of output connection 318. The ground connection (SOURCE) of power switch 316g may be connected to at least one of ground 304 and/or input connection 312. For example, the ground connection (SOURCE) of power switch 316g may be connected to a pin of input connection 312 corresponding to ground 304. Trigger signal line 319 may be connected to output connection 318. For example, trigger signal line 319 may be connected to trigger switch input 319aa of output connection 318.

A therapeutic tool connected to therapeutic tool/heating element outputs 320a may include heating element, as described herein. For example, the heating element may include a resistive heating element, which may be configured to receive the constant output power or the pulsed output power from power control system 310b and/or may heat based on the received power. The heating element may increase in temperature during the first time interval based on the constant output power until reaching a target temperature, as described herein. During the second time interval, a temperature of the heating element may be maintained within a target temperature range based on the pulsed output power, as described herein. For example, when the pulsed output power is high, the heating element may increase in temperature within the target temperature range, and/or when the pulsed output power is low, the heating element may decrease in temperature within the target temperature range.

Feedback resistor 324 may be connected to at least one of output connection 318 and/or input connection 312. For example, one side of feedback resistor 324 may be connected to a pin (e.g., pin 1) of output connection 318 and a pin (e.g., pin 4) of input connection 312, and a second side of feedback resistor 324 may be connected to a pin (e.g., pin 6, corresponding to feedback output 324a) of input connection 312. A power supply may receive a signal via feedback output 324a based on the current through feedback resistor 324. The power supply may turn on based on receiving the signal from feedback resistor 324.

For the purpose of illustration, upon the closing of a trigger switch connected to trigger switch input 319aa, power is supplied from a power supply to power supply input 302a of input connection 312, which supplies power to input power line 313. Input power line 313 supplies power to first power control circuit 316a and second power control circuit 316b (i.e., to second one-shot pulse generator circuit 316c and oscillator circuit 316d). First power control circuit 316a supplies constant output power during the first time interval, which is set based on the resistors 317a, 317b, and 317c. The output power from first power control circuit 316a is supplied to first logic gate 316e, which in turn supplies the constant output power to second logic gate 316f, and, since the trigger switch connected to trigger switch input 319aa is closed, second logic gate 316f, supplies the constant output power to output connection 318. Output connection 318 supplies the constant output power to a therapeutic tool connected to therapeutic tool/heating element outputs 320a, and the therapeutic tool supplies the constant output power to a heating element thereof to heat the heating element to a target temperature during the first time interval. After the first time interval, first power control circuit 316a stops supplying the constant output power.

During the second time interval following the first time interval (or during an entire time interval including both the first and second time interval), second one-shot pulse generator circuit 316c supplies constant output power to oscillator circuit 316d. The (second or entire) time interval during which second one-shot pulse generator circuit 316c supplies constant output power is set based on the resistors 317d, 317e, and 317f. Oscillator circuit 316d supplies pulsed output power for as long as second one-shot pulse generator circuit 316c supplies constant output power. The frequency of the pulsed output power from oscillator circuit 316d is set based on resistors 317g, 317h, and 317i. The output power from oscillator circuit 316d is supplied to first logic gate 316e, which in turn supplies the pulsed output power to second logic gate 316f during the second time interval (since the constant output power from first power control circuit 316a stopped after the first time interval). Since the trigger switch connected to trigger switch input 319aa is closed, second logic gate 316f supplies the pulsed output power to output connection 318. Output connection 318 supplies the pulsed output power to the therapeutic tool connected to therapeutic tool/heating element outputs 320a, and the therapeutic tool supplies the pulsed output power to the heating element to maintain the temperature of the heating element at a relatively constant temperature and/or within a target temperature range during the second time interval. After the second time interval, second one-shot pulse generator circuit 316c stops supplying power to oscillator circuit 316d, which, therefore, stops supplying the pulsed output power.

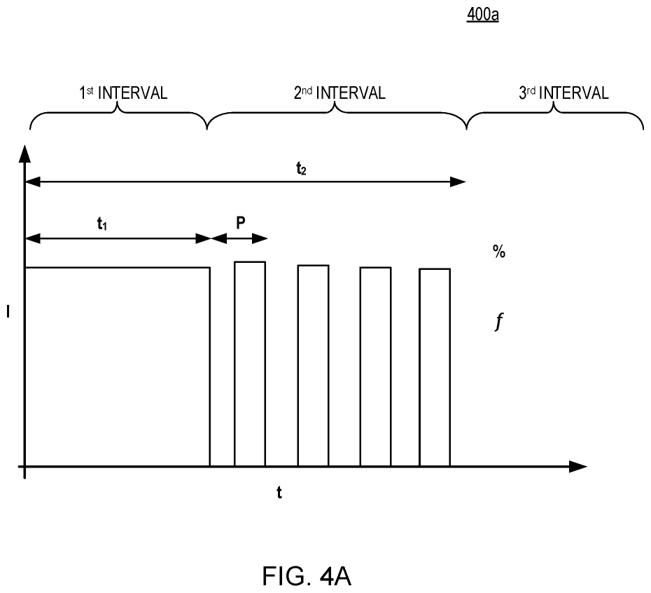

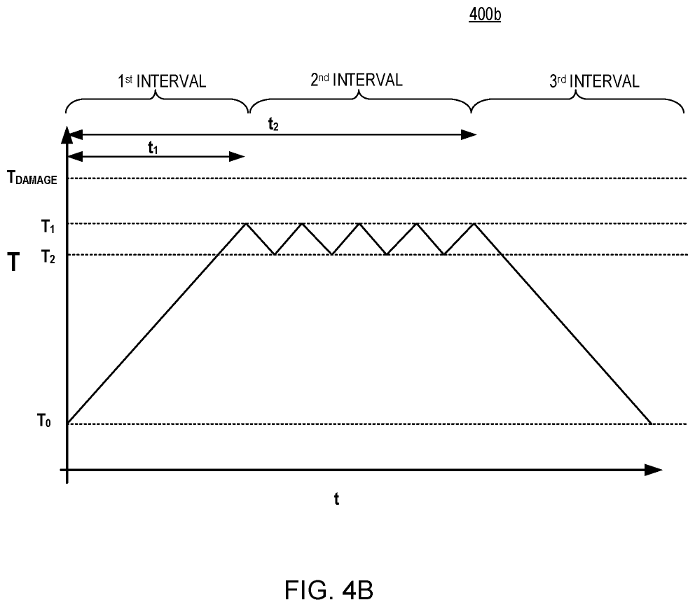

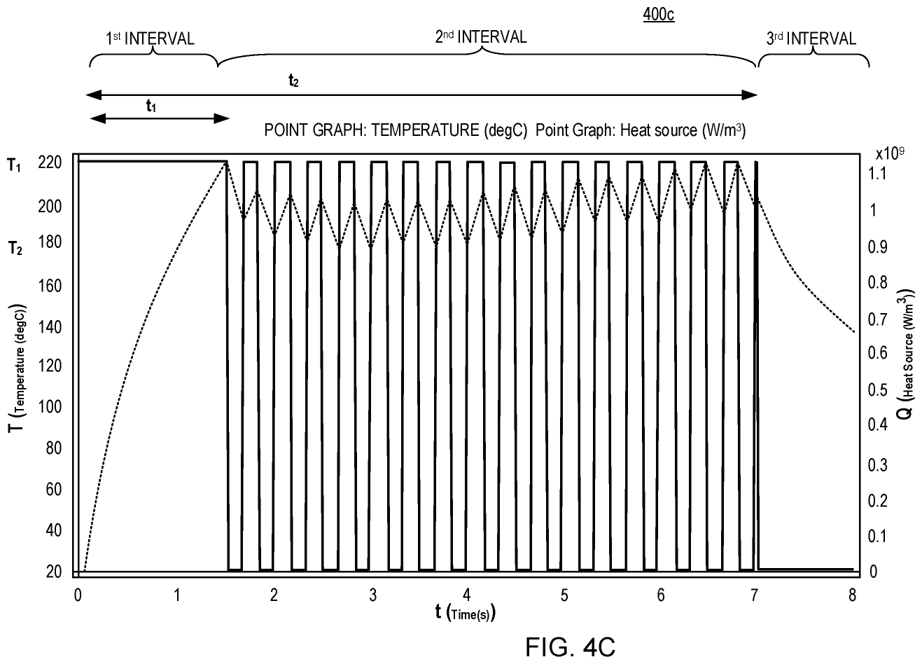

FIG. 4A is a graph 400a of current (I) versus time (t) of an exemplary implementation of non-limiting embodiments or aspects relating to a system and/or method for power control for a therapeutic tool, and FIG. 4B is a corresponding graph of temperature (T) versus time (t) of a heating element of the therapeutic tool that results from the current (I) flowing through the heating element. As shown in FIG. 4A, graph 400a has a vertical axis associated with current (I) and a horizontal axis associated with time (t). As shown in FIG. 4B, graph 400b has a vertical axis associated with temperature (T) and a horizontal axis associated with time (t).

During a first time interval (from 0 to t1), constant output power may be supplied by power control system 110, 210a, 210b, 310a, 310b, as described herein. For example, first power control circuit 116a, 316a may be connected to output connection 118, 218, 318 and/or configured to supply the constant output power during the first time interval to the output connection, as shown in FIG. 4A where current (I) is constant during the first interval. Consequently, heating element 122, 322 of therapeutic tool 120, 320 may heat to a target temperature T₁ as shown in FIG. 4B. The first time interval (t₁) may be selected based on the target temperature T₁ of heating element 122, 322. For example, a longer first time interval may result in heating of heating element 122, 322 to a higher temperature because the constant output power heats the heating element for a longer amount of time and at a steady rate. A shorter first time interval may result in heating of heating element 122, 322 to a lower temperature because the constant output power heats the heating element for a shorter amount of time.

During a second time interval (from time t₁ to time t₂), pulsed output power may be supplied by power control system 110, 210a, 210b, 310a, 310b, as described herein. For example, second power control circuit 116b, 316b may be connected to output connection 118, 218, 318 and/or configured to supply the pulsed output power during the second time interval to the output connection, as shown in FIG. 4A where current (I) is a periodic square wave having a duty cycle (%) and a frequency (f), each period (P) of which may include a first duration when the power is low and a second duration when the power is high, as described herein. Consequently, as shown in FIG. 4B, when the pulsed output power is low, heating element 122, 322 may decrease in temperature within the target temperature range between temperature T₁ and temperature T₂, and when the pulsed output power is high, heating element 122, 322 may increase in temperature within the target temperature range.

To maintain the temperature of heating element 122, 322 at a relatively constant temperature during the second time interval, the frequency (f) and/or duty cycle (%) may be selected so that cooling of the heating element during the first duration of low power is offset by heating of the heating element during the second duration of high power during each period (P), as described herein. To gradually increase the temperature of heating element 122, 322 during the second time interval, the frequency (f) and/or duty cycle (%) may be selected so that heating exceeds cooling during each period (P), as described herein. To gradually decrease the temperature of heating element 122, 322 during the second time interval, the frequency (f) and/or duty cycle (%) may be selected so that cooling exceeds heating during each period (P), as described herein.

As shown in FIG. 4B, the target temperature (T₁) during the first interval and/or the target temperature range (between T₁ and T₂) may be less than a damaging temperature (T_{damage}). For example, the damaging temperature (T_{damage}) may be the lowest of a temperature that may cause damage to therapeutic tool 120, 320, a temperature that may cause damage to collateral tissue, or a temperature that may cause excessive damage to the target tissue.

During a third time interval (after t₂), no power may be supplied by power control system 110, 210a, 210b, 310a, 310b, as described herein, for a predetermined period of time. For example, both first power control circuit 116a, 316a and second power control circuit 116b, 316b may cease supplying output power, as shown in FIG. 4A where current (I) is zero during the third time interval. Consequently, heating element 122, 322 of therapeutic tool 120, 320 may cool as a way to help mitigate excessive damage to the target tissue, collateral tissue damage, and/or damage to therapeutic tool 120, as shown in FIG. 4B. For example, heating element 122, 322 may cool to a baseline temperature (To), which may be the temperature of the environment surrounding heating element 122.

FIG. 4C is a graph 400c of temperature (T) and power density (Q) of a heating element versus time (t) of an exemplary implementation of non-limiting embodiments or aspects relating to a system and/or method for power control for a therapeutic tool. As shown in FIG. 4C, graph 400c has a left vertical axis associated with temperature (T), a right vertical axis associated with power density (Q), and a horizontal axis associated with time (t).

During a first time interval (from 0 to t1), constant output power may be supplied by power control system 110, 210a, 210b, 310a, 310b, as described herein. For example, first power control circuit 116a, 316a may be configured to supply the constant output power during the first time interval to heating element 122, 322 of therapeutic tool 120, 320, as shown in FIG. 4A where power density (Q) in heating element 122, 322 is constant during the first interval. Consequently, heating element 122, 322 may heat to a target temperature T₁. The first time interval (t₁) may be selected based on the target temperature T₁ of heating element 122, 322, as described herein. For example, a first time interval of 0.8 seconds may heat heating element 122 to 150° C., a first time interval of 1.6 seconds may heat heating element 122 to 200° C., or first time interval of 3.0 seconds may heat heating element 122 to 250° C.

During a second time interval (from time t₁ to time t₂), pulsed output power may be supplied by power control system 110, 210a, 210b, 310a, 310b, as described herein. For example, second power control circuit 116b, 316b may be configured to supply the pulsed output power during the second time interval to heating element 122, 322, as shown in FIG. 4C where power density (Q) is a periodic square wave, as described herein. Consequently, when the pulsed output power is low, heating element 122, 322 may decrease in temperature within the target temperature range between temperature T₁ and temperature T₂, and when the pulsed output power is high, heating element 122, 322 may increase in temperature within the target temperature range. For the purpose of illustration, as shown in FIG. 4C, the (average) temperature of heating element 122, 322 gradually decreases between the beginning of the second time interval (starting at about 1.7 seconds) and about 3 seconds, and the (average) temperature gradually increases from about 3 seconds to the end of the second time interval (ending at about 7 seconds).

During a third time interval (after t₂), no power may be supplied by power control system 110, 210a, 210b, 310a, 310b), as described herein. For example, both first power control circuit 116a, 316a and second power control circuit 116b, 316b may cease supplying output power, as shown in FIG. 4C where power density (Q) is zero during the third time interval. Consequently, heating element 122, 322 of therapeutic tool 120, 320 may cool as a way to help mitigate excessive damage to the target tissue, collateral tissue damage, and/or damage to therapeutic tool 120, as shown in FIG. 4C.

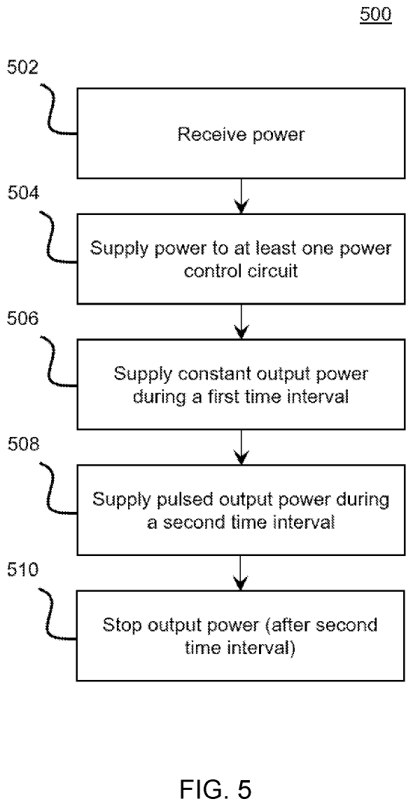

FIG. 5 is a flowchart of non-limiting embodiments or aspects of a process 500 for using a power control system for a therapeutic tool. One or more of the steps of process 500 may be performed (completely or partially) by power control system 110 (or one or more components thereof). One or more of the steps of process 500 may be performed (completely or partially) by another system, another device, another group of systems, or another group of devices, separate from or including power control system 110, such as power supply 102 and/or therapeutic tool 120.

As shown in FIG. 5, at step 502, process 500 may include receiving power, as described herein. For example, power control system 110 may receive power from power supply 102 via input connection 112, as described herein.

As shown in FIG. 5, at step 504, process 500 may include supplying power to at least one power control circuit, as described herein. For example, input connection 112 may supply power to at least one of circuit board 114, first power control circuit 116a, or second power control circuit 116b, as described herein. Input connection 112 may supply power to first power control circuit 116a and second power control circuit 116b, as described herein.

As shown in FIG. 5, at step 506, process 500 may include supplying constant output power during a first time interval, for example, to heat heating element 122 to a target temperature during the first time interval, as described herein. For example, first power control circuit 116a of power control system 110 may supply constant output power during the first time interval, as described herein. The first time interval may be selected based on a target temperature of heating element 122 of therapeutic tool 120, as described herein.

As shown in FIG. 5, at step 508, process 500 may include supplying pulsed output power during a second time interval following the first time interval, for example, to maintain the temperature of heating element 122 at a relatively constant temperature within a target temperature range during the second time interval, as described herein. For example, second power control circuit 116b of power control system 110 may supply pulsed output power during the second time interval, as described herein. The second time interval may be selected based on an average time for a procedure or portion thereof during which heat from heating element 122 will be used, as described herein. The pulsed output power may have a frequency and/or duty cycle selected to maintain (or gradually increase or gradually decrease) a temperature of heating element 122 of therapeutic tool 120 within a target temperature range, as described herein. The target temperature range may be a constant target temperature range, a gradually increasing target temperature range, or a gradually decreasing target temperature range, as described herein.

The constant output power or the pulsed output power may be received by output connection 118 of power control system 110, as described herein. The constant output power or the pulsed output power may be supplied from output connection 118 of power control system 110 to therapeutic tool 120 and/or heating element 122 thereof, as described herein.

As shown in FIG. 5, at step 510, process 500 may include stopping the output power, as described herein. For example, power control system 110 may not output power during a third time interval following the second time interval, as described herein. First power control circuit 116a may not output power, such as by stopping supply of the constant output power, after the first time interval. Second power control circuit 116b may not output power, such as by stopping supply of the pulsed output power, after the second time interval.

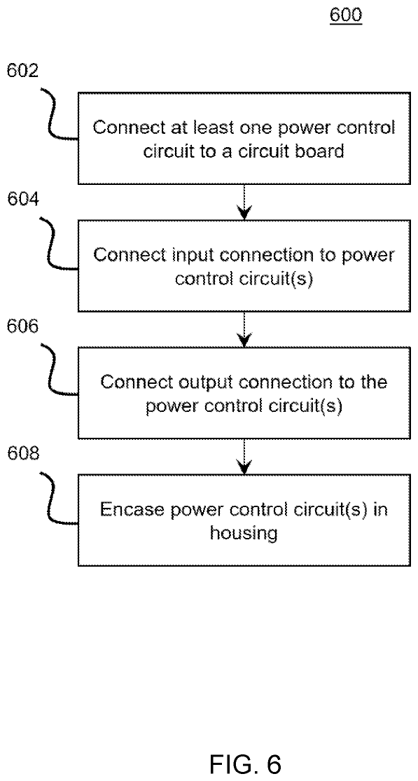

FIG. 6 is a flowchart of non-limiting embodiments or aspects of a process 600 for making a power control system for a therapeutic tool.

As shown in FIG. 6, at step 602, process 600 may include connecting at least one power control circuit to a circuit board, as described herein. For example, at least one of first power control circuit 116a, second power control circuit 116b, and/or logic gate(s) 117 may be connected to circuit board 114, as described herein.

First power control circuit 116a may include a first one-shot pulse generator circuit, as described herein. Second power control circuit 116b may include a second one-shot pulse generator circuit and an oscillator circuit, and the output of the second one-shot pulse generator circuit may be connected to an input of the oscillator circuit, as described herein.

Logic gate(s) 117 may be connected to circuit board 114, as described herein. For example, a first logic gate may be connected to circuit board 114, and/or the first logic gate may be connected to first power control circuit 116a and/or second power control circuit 116b, as described herein. A second logic gate may be connected to circuit board 114, and/or the second logic gate may be connected to the first logic gate and a switch, as described herein.

As shown in FIG. 6, at step 604, process 600 may include connecting an input connection to the power control circuit(s), as described herein. For example, input connection 112 may be connected to circuit board 114 and/or to first power control circuit 116a and second power control circuit 116b, as described herein.

Input connection 112 may be connected to a proximal end of a cable, as described herein. A distal end of the cable may be connected to circuit board 114, as described herein.

As shown in FIG. 6, at step 606, process 600 may include connecting an output connection to the power control circuit(s), as described herein. For example, output connection 118 may be connected to circuit board 114 and/or to first power control circuit 116a and second power control circuit 116b, as described herein. For example, output connection may be connected via circuit board 114 to logic gate(s) 117, which may be connected via circuit board 114 to first power control circuit 116a and second power control circuit 116b, as described herein.

As shown in FIG. 6, at step 608, process 600 may include encasing the power control circuit(s) in a housing, as described herein. For example, circuit board 114 and/or first power control circuit 116a and second power control circuit 116b may be encased in a housing, as described herein. At least one of input connection 112 and/or output connection 118 may be encased in the housing, as described herein. For example, circuit board 114 (having first power control circuit 116a, second power control circuit 116b, and logic gate(s) 117 connected thereto) and output connection 118 may be encased in the housing, as described herein. A cable may extend from the housing to input connection 112, as described herein.

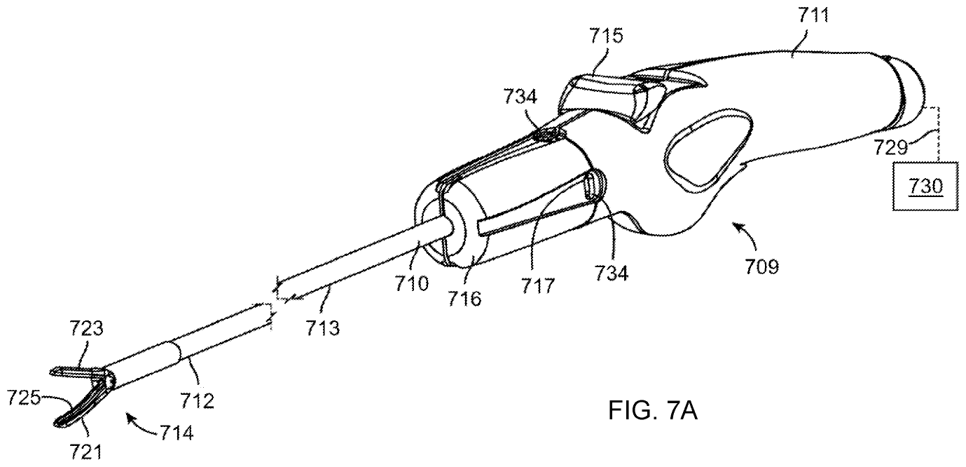

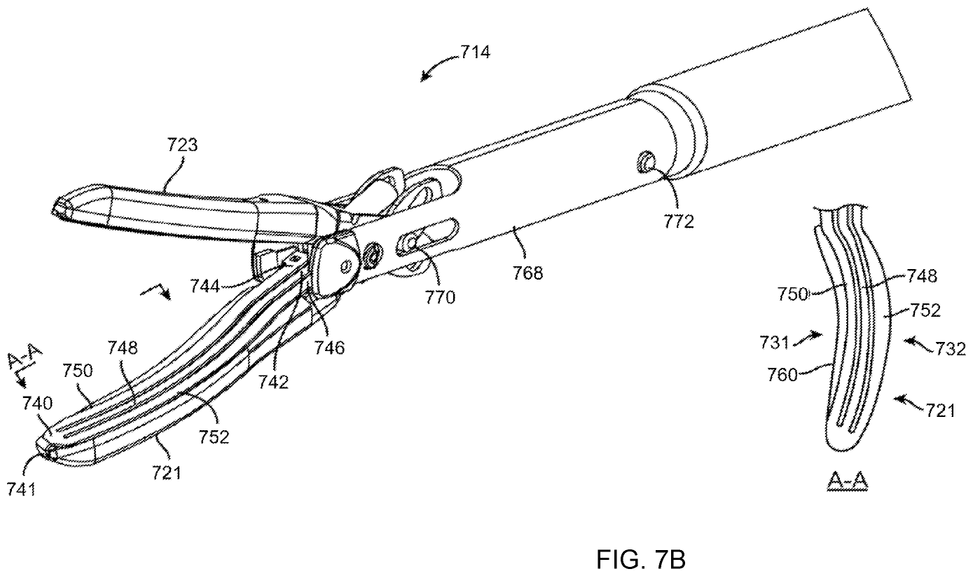

FIGS. 7A and 7B are diagrams of non-limiting embodiments or aspects of an exemplary therapeutic tool 709.

Therapeutic tool 709 may include handle 711, elongated body 713 having proximal end 710 and distal end 712, and surgical device/tool 714 located at distal end 712 of body 713. Proximal end 710 of elongated body 713 may be coupled to distal end 716 of handle 711. Elongated body 713 may be rigid, or alternatively, flexible. Handle 711 may include actuator 715 that may be coupled to surgical device 714 through a linkage (not shown) within a bore of elongated body 713 for controlling an operation of surgical device 714. Handle 711 and actuator 715 may be made from insulative material(s) such as plastic.

Surgical device 714 may include a pair of jaws 721, 723 for clamping, cutting, and/or sealing a vessel. For example, jaw 721 may include electrically conductive material 725 which faces towards opposing jaw 723. Jaw 723 may include an electrically conductive material which faces towards jaw 721. Electrically conductive material 725 may be in a form of an electrode and/or may be configured to selectively provide heat (and therefore act as a heating element) during use. As used with reference to FIGS. 7A and 7B, the term “electrode” may refer to a component that is for delivering energy, such as heat energy. Electrically conductive material 725 may be Ni-chrome, stainless steel, or other metals or alloys. Jaws 721, 723 may be configured to close in response to actuation (e.g., pressing, pulling, or pushing, etc.) of actuator 715, thereby clamping a vessel during use. Actuator 715 may be further actuated to cause electrically conductive material 725 to provide heat, thereby cutting and sealing the clamped vessel. For example, when actuator 715 is further actuated, electrically conductive material 725 may be electrically coupled, via cable 729, to DC source 730 (e.g., directly or via a power control system, such as power control system 110), which may provide a current to electrically conductive material (electrode) 725, thereby heating electrode 725. After the vessel is cut and sealed, actuator 715 may be de-actuated to stop the delivery of current to electrode 725 (and/or delivery of the current may automatically be stopped by power control system 110, such as during a third time interval), and actuator 715 may be further de-actuated to open jaws 721, 723. The mechanical linkage for translating operation of actuator 715 into closing and opening of jaws 721, 723 may be implemented using cables, shafts, gears, or any of other suitable mechanical devices.

Handle 711 also may include a plurality of electrical contact terminals 717 in respective ports 734 near distal end 716 of handle 711. Contact terminals 717 may be electrically coupled to electrically conductive material 725 at surgical device 714, and/or may be configured (e.g., shaped, sized, and positioned) for receiving energy from a power source. Each contact terminal 717 may be electrically connected to electrode 725 via electrical line that may be housed within a wall of elongated body 713, or that may be in a form of a cable that is housed within the bore of elongated body 713. Elongated body 713 may include an outer layer of bioinert electrically insulative material. Instead of being located inside port 734, contact terminal 717 may be in a form of a ring located and exposed near distal end 716 of handle 711.

The linkage that mechanically couples jaws 721, 723 to actuator 715 may be electrically insulated, for example, by silicone rubber, ceramic or other suitable non-electrically conductive material. This may assure that high frequency energy supplied to contact terminal 717 is conducted along the electric line housed by body 713 to electrically conductive material (electrode) 725 at jaw 721 (and/or electrode at jaw 723). Body 713 may not include an electric line for coupling contact terminal 717 to electrode 725. Instead, linkage that mechanically couples jaws 721, 723 to actuator 715 may be electrically conductive, and may be used to couple electrical energy received at contact terminal 717 to electrode 725 at jaw 721 (and/or electrode at jaw 723). For example, the linkage may be slidably coupled to contact terminal 717.

Connection ports 734 may be disposed about the periphery of handle 711 near its distal end 716. Each such connection port 734 may be configured to selectively receive the tip of an electrosurgical probe, thereby allowing the respective contact terminal 717 to electrically connect such a probe through the electrical line housed in body 713 (or through the mechanical linkage, such as an actuating rod, within body 713 if the linkage is electrically conductive) to electrically conductive material 725 at the distal end. By providing a plurality of ports 734 circumferentially about the distal portion of handle 711, therapeutic tool 709 may allow a probe to make contact with terminal 717 no matter how elongated body 713 is oriented about is longitudinal axis. The actuating rod may be mechanically linked to actuator 715 to slidably translate within elongated body 713 in response to fore and aft movements of actuator 715. Translational movement of the actuating rod may be linked to jaws 721, 723 to open and close the jaws in response to movement of actuator 715. Providing port(s) 734 and contact terminal(s) 717 in port(s) 734 in this exemplary configuration may prevent unintentional contact of the contact terminal(s) by the user during use. Instead of (only) providing port(s) 734 at handle 711, (at least some) port(s) 734 may be provided at elongated body 713.

Electrically conductive material 725 may form heating element (electrode) 740 that is disposed on a surface of jaw 721. Heating element 740 may include two outer portions 750, 752, and inner (middle) portion 748. Outer portions 750, 752 may have respective outer terminals 744, 746 at their ends, and middle portion 748 may have inner terminal 742 at its end. Thus, portions 748, 750, 752 may form an electrical heater circuit between inner terminal 742 and outer terminals 744, 746. Outer portions 750, 752 and inner portion 748 may function as an electrode that is configured to deliver heat. For example, inner terminal 742 of electrode 740 may be electrically coupled to a first terminal of DC source 730 (and/or output connection 118 of power control system 110), and outer terminals 744, 746 of electrode 740 may be electrically coupled to a second terminal of DC source 730 (and/or output connection 118 of power control system 110), thereby allowing electrode 740 to receive and conduct DC energy (for cutting and/or welding tissue). Heating element 740 may be formed using a single, flat sheet of electrically conductive material (e.g., Ni-chrome alloy, such as stainless steel at an outer layer, and Ni-chrome at an inner layer), which may have reliability, manufacturing, and/or cost advantages and/or may reduce the likelihood of tissue build up and entrapment during use by minimizing crevices into which tissue can migrate. Distal end 741 of heater element 740 may be disposed beyond the distal end of jaw 721 (at the distal tip) to serve as an exposed electrode, which may allow cauterization of tissue by electrical energy to be performed using the distal tip of jaw 721.

The jaw-operating mechanism and linkage of such mechanism, for example, to an actuating rod, may be supported in metal housing 768 that includes metal sliding pin 770 and attachment pin 772, all of which may be covered with an insulating layer (not shown) of flexible material such as silicone rubber, or the like, to shield/protect adjacent tissue from moving parts and/or from electrical energy within the instrument. For example, such an insulating cover may retain the sliding and attachment pins 770, 772 in place to obviate the need for more expensive fasteners and mechanisms.

During use in a first mode of operation, current from DC source 730 (e.g., via power control system 110) may be conducted through inner terminal 742 and/or flow in inner (middle) portion 748 of heating element 740 and in parallel through the dual outer portions 750, 752 of heating element 740 to outer terminals 744, 746. For example, for heater portions 748, 750, 752 of equal thicknesses and equal widths, current density in inner (middle) portion 748 may be twice as high as the current density in each of outer portions 750, 752 in response to electrical heater signal applied between inner terminal 742 and outer terminals 744, 746. Current densities in inner and outer portions 748, 750, 752 may be altered (for example, by altering the relative widths of the heater portions, by altering resistances through selection of different materials, by altering both the widths and resistances, etc.) to alter the operating temperatures thereof in response to applied electrical heater signals. In operation, outer portions 750, 752 may operate at a temperature sufficient to weld a tissue structure grasped between jaws 721, 723, and inner portion 748 may operate at a higher temperature sufficient to sever the grasped tissue structure intermediate of the welded segments.

The jaw assembly may have concave side 731 and convex side 732. In one method of use, while the jaw assembly is used to cut a side branch vessel, the jaw assembly may be oriented so that its concave side 731 faces towards the main vessel. For example, an endoscope or viewing device may be placed next to the jaw assembly with the endoscope or viewing device viewing concave side 731 of the jaw assembly. This may allow the user to better visualize the tip of the jaw assembly. Such configuration may also provide a safety benefit by allowing the user to know where the tips are during a vessel cutting procedure. Exposed outer portion 752 may be on convex side 732 of the jaw assembly while protrusion 760 may be on concave side 731 of the jaw assembly. The concavity may provide extra spacing to further protect the main vessel when the side branch vessel is grasped. Furthermore, exposed outer portion 752 on convex side 732 may create a protrusion that makes it easier to contact the wall of the tunnel with exposed outer portion 752 to address bleeding. Protrusion 760 may be on the convex side 732 of the jaw assembly while exposed outer portion 752 may be on concave side 731. Consequently, during use, convex side 732 of the jaw assembly may be oriented towards the main vessel, thereby ensuring that the tips of the jaw assembly are separated from the main vessel to enhance protection by preventing the tip of the jaw assembly from touching or injuring the main vessel.

The temperature to which the heating elements on the jaws rise may also affect the preferred force applied, as well as the duration of the weld. For example, a range of temperatures at which human tissue may be welded may be 50 to 90° C., while severing may occur at temperatures of 100° C. and above. Consequently, if the exemplary jaws apply a clamping force of between 1-3 pounds on tissue and the welding and severing heating elements are energized to these temperature ranges, respectively, a duration of a weld may be between 1-5 seconds. If the clamp duration is too short, the weld may not be effective and the tissue may be less likely to completely sever, while an excessive duration above, for example, 5 seconds may tend to char tissue.

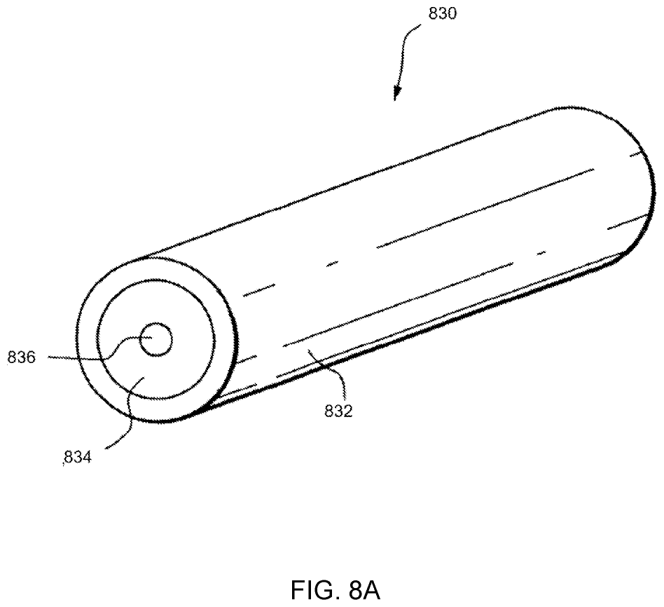

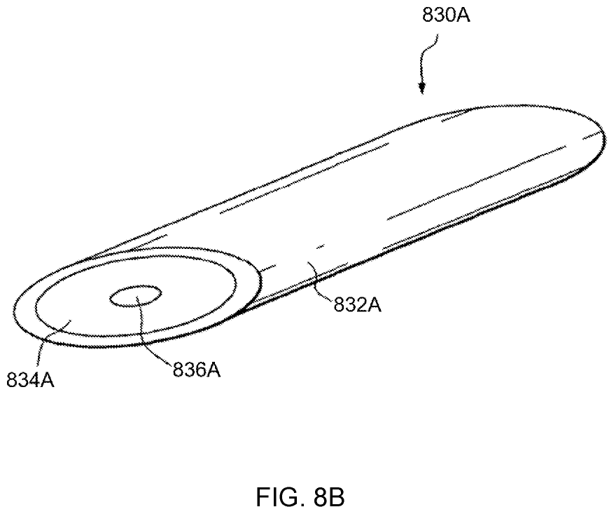

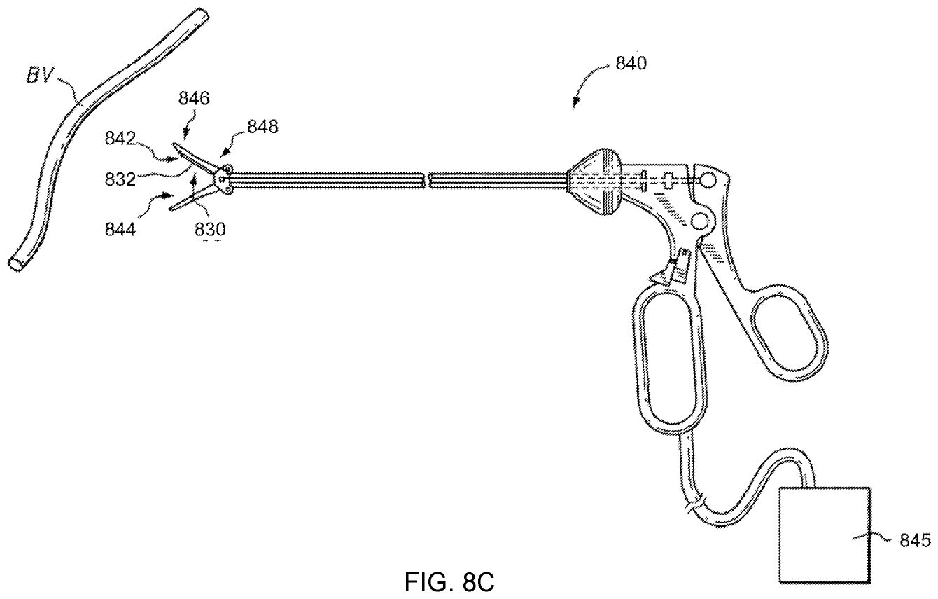

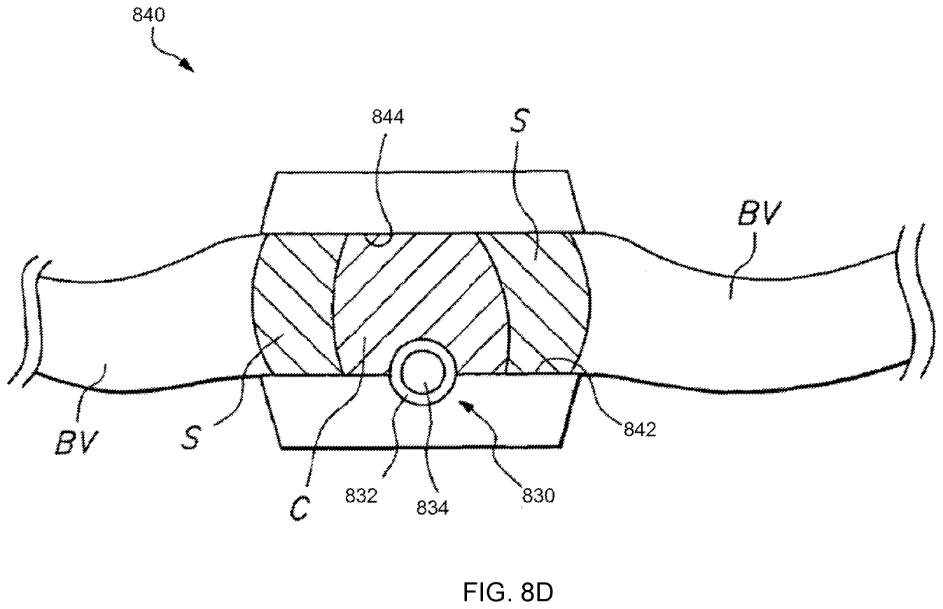

FIGS. 8A-8D are diagrams of non-limiting embodiments or aspects of an exemplary therapeutic tool 840.

FIG. 8A illustrates heater 830. For example, heater 830 may include tubular shaped resistance heating element 832 as its exterior surface. Heating element 832 may be made of any suitable resistance material, including, but not limited to, metal alloys such as Nichrome™ or Inconel™. Within the interior of heating element 832, a high resistance, electrically insulating, high thermal conductivity core material 834 may be disposed. For example, core material 834 may be made of a ceramic, and may include materials such as magnesium oxide, boron nitride, or aluminum nitride. Core material 834 may simply include air or any other gas. Heater 830 may be formed by metalizing a ceramic rod.

FIG. 8B shows an oval shaped embodiment or aspect of the heater, which may offer the advantage of increased tissue contact surface area (compared to FIG. 8A). Elements 830A, 832A, 834A, and 836A in FIG. 8B may correspond respectively to elements 830, 832, 834, and 836 in FIG. 8A.

Temperature sensing element 836 may also be included. For example, temperature sensing element 836 may or may not be present, as desired. Temperature sensing element 836 may include a thermocouple, a thermistor, a positive temperature coefficient (PTC) element or a negative temperature coefficient (NTC) element. For example, a PTC material such as tungsten wire may be useful as it may be incorporated within heater element 832 during manufacturing. Other suitable PTC materials may include an alloy and/or iron.

The DC resistance of tubular shaped heating element 832 may be less than that of the surrounding body tissue. For example, the resistance of tubular shaped heating element 832 may be less than 10 ohms.

The outer diameter of tubular heating element 832 may be between 0.35 mm and 0.55 mm, and the wall thickness may be about 0.0254 mm.

The operation of heater 830 is illustrated in FIGS. 8C and 8D. Referring to FIG. 8C, a therapeutic tool 840 may be provided. For example, therapeutic tool 840 may include a pair of ligating shears (as shown) and/or may include a pair of tweezers or forceps, or any other device adapted to grasp onto and hold tissue between a pair of arms or jaws.

Therapeutic tool 840 may include a pair of opposing working surfaces 842 and 844. For example, at least one heater 830 may be positioned on the surface of one or more of the working surfaces. For the purpose of illustration, heater 830 may be positioned on the surface of working surface 842.

Therapeutic tool 840 may be used to cut or seal tissue by first grasping the tissue between two opposing working surfaces 842 and 844 and passing current (e.g., from power control system 110) through the tubular heating element 832, thereby causing heating of the tissue surrounding heater 830. For example, the tissue may be mechanically squeezed between opposing working surfaces 842 and 844 while current is passing through tubular heating element 832, so as to better “seal” adjacent tissues together.

Therapeutic tool 840 may further include electrical leads 846 and 848 connected to tubular heating element 832 at different points along its length. Power source 845 may be electrically connected (e.g., directly or via power control system 110) to leads 846 and 848 so as to conduct current through tubular heating element 832, thereby heating tubular heating element 832. The current passing through tubular heating element 832 may not exceed 10 A. Power source 845 may alternately be a constant current power source, a constant voltage power source, a temperature feedback control power source, and/or power source 845 may provide constant power to a power control system (e.g., power control system 110), which may provide power to therapeutic tool 840, as described herein.

FIG. 8C also shows a blood vessel BV which can be grasped onto as shown in the schematic view of FIG. 8D. For example, as seen in FIG. 8D, blood vessel BV may be held between working surfaces 842 and 844 of therapeutic tool 840. As can be seen, the portion of tissue closest to heater 830 may be heated in excess of 100° Celsius such that the tissue structure may be broken down, forming a cut zone C. On either side of cut zone C, where the tissue is farther away from heater 830, the tissue may only be heated to a temperature between 50° and 90° Celsius, thus forming a seal zone S. The mechanical pressure exerted by forcing working surfaces 842 and 844 together against blood vessel BV may further assist in tissue sealing.

Certain therapeutic tools 120, such as DC powered EVH systems, may require safeguards in order to limit the amount of power delivered to at least one heating element 122 thereof, such as a resistive heating element located on a jaw, to prevent excessive temperatures. Power control system 110 may modulate power delivery to such a therapeutic tool 120 to maintain a relatively constant temperature at the heating element 122 of the device. As such, power control system 110 may maintain a safe and/or effective temperature and/or prevent overheating, which may reduce damage to therapeutic tool 120 and/or may make the medical procedure safer for the patient while maintaining effectiveness.

Power control system 110 may interrupt power delivery from power supply 102 to therapeutic tool 120 and/or regulate power to therapeutic tool 120 in three time intervals. During a first time interval (e.g., t₀ to t₁) constant output power may be supplied to therapeutic tool 120. For example, therapeutic tool 120 may be powered constantly and heating element(s) 122 thereof may increase in temperature as it would without power control system 110. The first time interval may be a few seconds (e.g., 2-5 seconds) to avoid reaching dangerous or destructive temperatures. During a second time interval (e.g., t₁ to t₂), power control system 110 may interrupt the power from power supply 102 to therapeutic tool 120 by rapidly cutting power to therapeutic tool 120 and supplying pulses of power to therapeutic tool 120. For example, such pulses may occur at a rate of approximately 6-12 Hz. The time between such pulses may allow heating element 122 to cool slightly before power returns by way of the next pulse, and this may enable maintaining a relatively constant temperature of heating element 122 that can be controlled. The second time interval may be longer than the first. For example, the second time interval may be 15-20 seconds to allow for performance of a medical procedure or portion thereof, such as proper cutting and/or sealing of a vessel during an EVH procedure. During a third time interval (e.g., after t₂), power control system 110 may shut off power to therapeutic tool 120. For example, after the second time interval is complete, power to therapeutic tool 120 may be stopped completely to prevent heating of heating element 122 for too long.

The times t₁ and t₂, the frequency (f), and/or the duty cycle (%) of the pulsed power may be selected as needed for a particular therapeutic tool 120, for a particular procedure or portion thereof during which heat from heating element 122 will be used, and/or for a particular type of target tissue. For example, adjusting t₁ may affect the target temperature that is reached during the first time interval and/or affect the range of temperatures that will be maintained during the second time interval. As t₁ increases, heating element 122 of therapeutic tool 120 may heat longer (before constant power is stopped and pulsed power is started), which may increase the temperature of heating element 122. As t₁ decreases, constant power delivery will be stopped sooner (and pulsed power will be started sooner), which may decrease the temperature of heating element 122. Adjusting t₂ may affect how long a user (e.g., a clinician) has to perform a procedure or portion thereof during which heat from heating element 122 will be used. Adjusting the frequency and/or duty cycle of the pulsed power may affect how stably the temperature of the heating element may be maintained during the second time interval. For example, longer pauses between pulses may allow the heating element of therapeutic tool 120 to cool more and/or make the temperature thereof less stable.

For the purpose of illustration and not limitation, Table 1 shows the average time for performance of a portion of an EVH procedure that includes cutting and sealing a vessel when power control system 110 is not used with an exemplary therapeutic tool 120, a Vasoview Hemopro 2 Endoscopic Vessel Harvesting System, and when power control system 110 is used with the exemplary therapeutic tool 120. As shown in Table 1, the procedures may be effectively performed in the same or a similar amount of time with the power control system 110 as without power control system 110, and this may demonstrate that the effectiveness may be maintained, while safety may be improved, as described herein.

TABLE 1

| | Time(sec) to | Time(sec) to | |
|---|---|---|---|
| | cut/seal without | cut/seal with | Sample |
| Tissue | Power Control System | Power Control System | Size |
| All | 4.8 | 4.7 | 10 |
| Blood Vessels | 4 | 3.8 | 5 |
| Vessels in | 7 | 7 | 2 |
| fascia | | | |
| Tendon | 4.7 | 4.7 | 3 |

Power control system 110 may allow a therapeutic tool 120 to be safely and effectively regulated while remaining consistent and dependable. For example, power control system 110 may allow the temperature of heating element 122 of therapeutic tool 120 to be more consistent, constant, and well-regulated to avoid overheating, may enable power to therapeutic tool 120 to be shut off at a reliable and consistent time, may allow the user to have plenty of time for performing a procedure or portion thereof (such as cutting and sealing a vessel during an EVH procedure), and may prevent “down time” before the device can be activated again after a procedure. For example, when therapeutic tool 120 is an EVH device, the power control system 110 may provide a more consistent and constant temperature of a resistive heating element located on a jaw, resulting in a more repeatable, uniform heating range, which produces better vessel cuts and seals and better durability of the jaws of the EVH device.

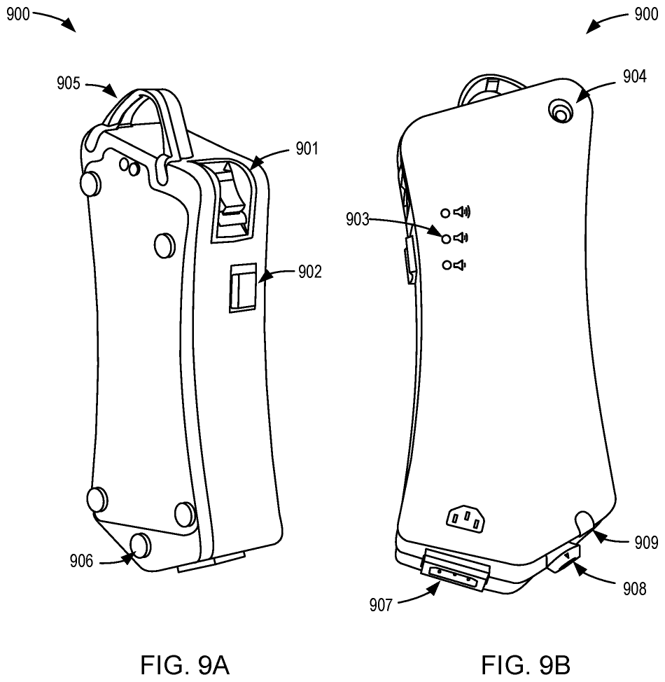

FIGS. 9A and 9B are diagrams of an exemplary implementation 900 of non-limiting embodiments or aspects relating to a power control system for a therapeutic tool. As shown in FIGS. 9A and 9B, implementation 900 may include power supply switch 901, volume switch 902, volume setting indicator 903, power indicator 904, hanging element 905, non-slip footings 906, power cord connection 907, output connection 908, and/or connection indicator 909.

As shown in FIGS. 9A and 9B, a power control system (e.g., 110) may be incorporated within a power supply (e.g., 102). For example, implementation 900 may be the same as or similar to power control system 110 and power supply 102, as described herein (e.g., as shown in FIGS. 1E-1F).

For example, implementation 900 may include any suitable power supply. For example, implementation 900 may include at least one device and/or a component thereof configured to supply power. Power cord connection 907 may include at least one connector for connecting a power cord to a power source (e.g., a power grid, a battery, or any combination thereof). For example, the power cord may be connected to a wall outlet (e.g., plug) connected to the power grid (e.g., mains electric power utility power, domestic power, and/or the like). Implementation 900 may convert AC power (e.g., from the power grid) to DC power (e.g., suitable for a therapeutic tool and/or power control system, as described herein). For example, implementation 900 may receive AC power from the power grid (e.g., 120 VAC and/or 240 VAC) and convert the AC power to DC power to supply the DC power as output (e.g., 5 amperes (A) at 5.5 volts DC (VDC)) for the power control system (e.g., 110) incorporated in implementation 900.

Power supply switch 901 may include at least one switch for turning on and/or off the power supply of implementation 900. For example, when power supply switch 901 is closed, the power supply of implementation 900 may be turned on, and when power supply switch 901 is open, the power supply of implementation 900 may be turned off. Power indicator 904 may indicate whether the power supply is on or off. For example, power indicator 904 may include a visual indicator (e.g., a light, such as a light emitting diodes (LED)). For example, power indicator 904 may be on (e.g., LED illuminating) when the power supply is on (e.g., power supply switch 901 is switched on and/or closed). Power indicator 904 may be off (e.g., LED not illuminating) when the power supply is off (e.g., power supply switch 901 is switched off and/or open).

Volume switch 902 may include at least one switch to adjust a volume of an audio indicator of implementation 900, as described herein. For example, volume switch 902 may be configured to allow for switching between at least to volume settings. Volume setting indicator 903 may indicate which volume setting is selected. For the purpose of illustration, as shown in FIGS. 9A-9B, volume switch 902 may be configured to allow for switching between three volume settings (e.g., low, medium, and high volume), and volume setting indicator 903 may include three visual indicators (e.g., lights, such as LEDs) to indicate which volume setting is selected (e.g., a bottom LED on to indicate a low volume setting, a middle LED on to indicate a medium volume setting, and a top LED on to indicate a high volume setting).

Hanging element 905 may include at least one element configured to allow implementation 900 to be hung (e.g., from a hook, peg, protrusion, and/or the like). For example, hanging element 905 may include at least one of a hook, a loop, a hanger, and/or the like.

Non-slip footings 906 may include at least one element configured to prevent implementation 900 from slipping when resting on a surface (e.g., a table, a countertop, a cart, a floor, and/or the like). For example, each non-slip footings 906 may include at least one of a pad, a bumper, a protrusion, an adhesive patch, and/or the like.

Output connection 908 may be configured to supply power from implementation 900 (e.g., the power control system thereof) to a therapeutic tool, as described herein. For example, output connection 908 may be the same as or similar to output connection 118. For example, output connection 908 may include any suitable electrical connection to connect implementation 900 to a therapeutic tool and/or a component thereof, as described herein. Connection indicator 909 may indicate whether the therapeutic tool is connected (e.g., properly connected). For example, connection indicator 909 may include a visual indicator (e.g., a light, such as an LED). For example, connection indicator 909 may be on (e.g., LED illuminating) when the implementation 900 is properly connected to the therapeutic tool. Connection indicator 909 may be off (e.g., LED not illuminating) when the implementation 900 is not properly connected to the therapeutic tool.

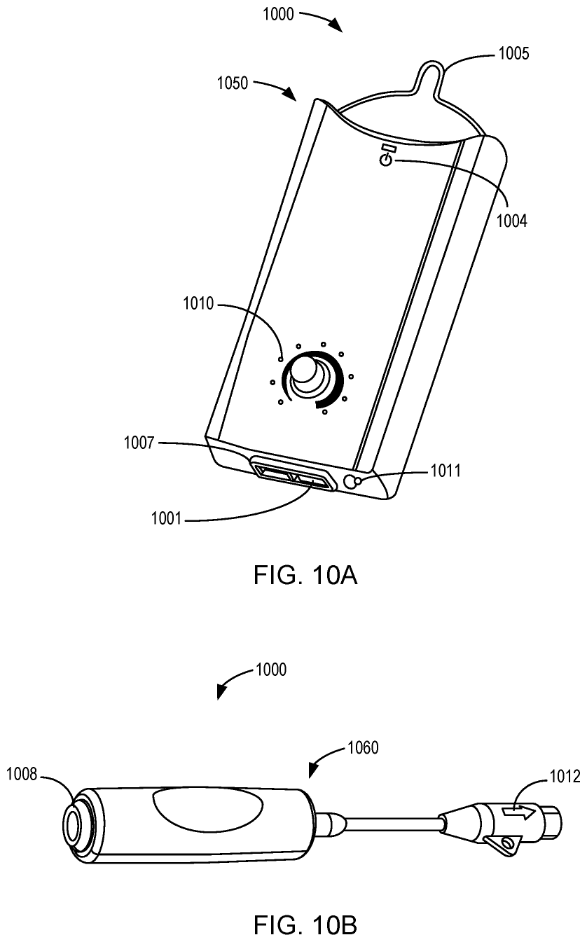

FIGS. 10A and 10B are diagrams of an exemplary implementation 1000 of non-limiting embodiments or aspects relating to a power control system for a therapeutic tool. As shown in FIGS. 10A-10B, implementation 1000 may include power supply 1050 and power control system 1060. Power supply 1050 may include power supply switch 1001, power indicator 1004, hanging element 1005, power cord connection 1007, power setting element 1010, and power control system connection 1011. Power control system 1060 may include input connection 1012 and output connection 1008. In some non-limiting embodiments or aspects, power supply 1050 may be the same as or similar to power supply 102. In some non-limiting embodiments or aspects, power control system 1060 may be the same as or similar to power control system 110.

As shown in FIGS. 10A and 10B, power control system 1060 may be separate from power supply 1050. For example, implementation 1000 may be the same as or similar to power control system 110 and power supply 102, as described herein (e.g., as shown in FIGS. 1A-1B).

Power supply 1050 may include any suitable power supply. For example, power supply 1050 may include at least one device and/or a component thereof configured to supply power. Power cord connection 1007 may include at least one connector for connecting a power cord to a power source (e.g., a power grid, a battery, or any combination thereof). For example, the power cord may be connected to a wall outlet (e.g., plug) connected to the power grid (e.g., mains electric power utility power, domestic power, and/or the like). Power supply 1050 may convert AC power (e.g., from the power grid) to DC power (e.g., suitable for a therapeutic tool and/or power control system 1060, as described herein). For example, power supply 1050 may receive AC power from the power grid (e.g., 120 VAC and/or 240 VAC) and convert the AC power to DC power to supply the DC power as output (e.g., 5 amperes (A) at 5.5 volts DC (VDC)) to power control system 1060 (e.g., via power control system connection 1011).

Power supply switch 1001 may include at least one switch for turning on and/or off power supply 1050. For example, when power supply switch 1001 is closed, power supply 1050 may be turned on, and when power supply switch 1001 is open, power supply 1050 may be turned off. Power indicator 1004 may indicate whether power supply 1050 is on or off. For example, power indicator 1004 may include a visual indicator (e.g., a light, such as an LED). For example, power indicator 1004 may be on (e.g., LED illuminating) when power supply 1050 is on (e.g., power supply switch 1001 is switched on and/or closed). Power indicator 1004 may be off (e.g., LED not illuminating) when power supply 1050 is off (e.g., power supply switch 1001 is switched off and/or open).

Power setting element 1010 may include at least one adjustable element (e.g., knob, switch, button, and/or the like) to adjust an output power (e.g., an amplitude of the current of the output power) of power supply 1050. For example, turning up power setting element 1010 (e.g., a knob) may increase the output power (e.g., the amplitude of the current of the output power), and turning down power setting element 1010 (e.g., a knob) may decrease the output power (e.g., the amplitude of the current of the output power).

Hanging element 1005 may include at least one element configured to allow power supply 1050 to be hung (e.g., from a hook, peg, protrusion, and/or the like). For example, hanging element 1005 may include at least one of a hook, a loop, a hanger, and/or the like.

Input connection 1012 of power control system 1060 may be configured to receive power from power supply 1050 (e.g., via power control system connection 1011 of power supply 1050). For example, input connection 1012 may include any suitable electrical connection to connect power control system 1060 to power control system connection 1011 of power supply 1050, as described herein. For example, input connection 1012 may be the same as or similar to input connection 112.

Output connection 1008 of power control system 1060 may be configured to supply power from power control system 1060 to a therapeutic tool, as described herein. For example, output connection 1008 may be the same as or similar to output connection 118. For example, output connection 1008 may include any suitable electrical connection to connect power control system 1060 to a therapeutic tool and/or a component thereof, as described herein.

Although the disclosed subject matter has been described in detail for the purpose of illustration based on what is currently considered to be the most practical and preferred embodiments or aspects, it is to be understood that such detail is solely for that purpose and that the disclosed subject matter is not limited to the disclosed embodiments or aspects, but, on the contrary, is intended to cover modifications and equivalent arrangements that are within the spirit and scope of the appended claims. For example, it is to be understood that the presently disclosed subject matter contemplates that, to the extent possible, one or more features of any embodiment or aspect can be combined with one or more features of any other embodiment or aspect.

What is claimed is:

1. An endoscopic vessel harvesting system comprising a direct current power control system connected to provide controlled power to a therapeutic tool comprising a heating element, the direct current power control system comprising:
an input connection configured to receive power from a power supply;
a first power control circuit connected to the input connection, the first power control circuit configured to supply constant output power to the heating element during a first time interval to heat the heating element to a target temperature;
a second power control circuit connected to the input connection, the second power control circuit configured to supply pulsed output power to the heating element during a second time interval following the first time interval to maintain a temperature of the heating element within a target temperature range; and
an output connection connected to the first power control circuit and the second power control circuit, the output connection configured to receive the constant output power from the first power control circuit and the pulsed output power from the second power control circuit to supply controlled power to the heating element of the therapeutic tool, wherein supplying of controlled power to the heating element comprises, in sequence, supplying the constant output power during the first time interval and then the pulsed output power during the second time interval to the heating element followed by a third time interval during which no power is supplied to the heating element to allow heat to dissipate from the heating element.

2. The endoscopic vessel harvesting system of claim 1, wherein the first power control circuit comprises a first one-shot pulse generator circuit.

3. The endoscopic vessel harvesting system of claim 1, wherein the first time interval comprises a time interval greater than or equal to 2 seconds and less than or equal to 10 seconds so that the heating element heats to the target temperature effective for performing an endoscopic vessel harvesting procedure.

4. The endoscopic vessel harvesting system of claim 3, wherein the target temperature effective for performing the endoscopic vessel harvesting procedure comprises a first temperature sufficient to at least one of cut, cauterize, or weld target tissue.

5. The endoscopic vessel harvesting system of claim 1, wherein the second power control circuit comprises a second one-shot pulse generator circuit and an oscillator circuit, wherein an output of the second one-shot pulse generator circuit is connected to an input of the oscillator circuit.

6. The endoscopic vessel harvesting system of claim 1, wherein the second time interval comprises a time interval greater than or equal to 5 seconds and less than or equal to 20 seconds.

7. The endoscopic vessel harvesting system of claim 6, wherein the second time interval is selected based on an average time to at least one of cut, cauterize, or weld target tissue.

8. The endoscopic vessel harvesting system of claim 1, wherein the pulsed output power has a frequency of greater than or equal to 2.5 Hz and less than or equal to 12 Hz.

9. The endoscopic vessel harvesting system of claim 8, wherein the frequency is selected to maintain the temperature of the heating element at the target temperature range effective for performing an endoscopic vessel harvesting procedure.

10. The endoscopic vessel harvesting system of claim 1, further comprising:
a circuit board comprising the first power control circuit and the second power control circuit;
a housing containing the circuit board and the output connection; and
a cable extending from the housing, the cable having a proximal end connected to the input connection and a distal end connected to the circuit board.

11. The endoscopic vessel harvesting system of claim 1, further comprising:
a first logic gate connected to the first power control circuit and the second power control circuit, wherein the first logic gate is configured to output only one of the constant output power from the first power control circuit or the pulsed output power from the second power control circuit at a time; and
a second logic gate connected to the first logic gate and a switch, wherein the second logic gate is configured to output the constant output power or the pulsed output power from the first logic gate only after the switch is closed.

12. A method for providing controlled power via a direct current power control system of an endoscopic vessel harvesting device so as to provide controlled power to a heating element of a therapeutic tool of the endoscopic vessel harvesting device, the method comprising:
receiving, at an input connection of the direct current power control system, power from a power supply;
supplying power from the input connection to a first power control circuit and a second power control circuit of the direct current power control system,
supplying constant output power from the first power control circuit during a first time interval to heat the heating element to a target temperature;
supplying pulsed output power from the second power control circuit during a second time interval following the first time interval to maintain a temperature of the heating element within a target temperature range;
receiving, at an output connection of the direct current power control system, the constant output power from the first power control circuit or the pulsed output power from the second power control circuit;
supplying the constant output power or the pulsed output power from the output connection to the therapeutic tool; and
supplying no power to the heating element during a third time interval following the second time interval to allow heat to dissipate from the heating element.

13. The method of claim 12, wherein the first time interval comprises a time interval greater than or equal to 2 seconds and less than or equal to 10 seconds so that the heating element heats to the target temperature effective for performing an endoscopic vessel harvesting procedure.

14. The method of claim 13, wherein the target temperature effective for performing the endoscopic vessel harvesting procedure comprises a first temperature sufficient to at least one of cut, cauterize, or weld target tissue.

15. The method of claim 12, wherein the second time interval comprises a time interval greater than or equal to 5 seconds and less than or equal to 20 seconds.

16. The method of claim 15, wherein the second time interval is selected based on an average time to at least one of cut, cauterize, or weld target tissue.

17. The method of claim 12, wherein the pulsed output power has a frequency of greater than or equal to 2.5 Hz and less than or equal to 12 Hz.

18. The method of claim 17, wherein the frequency is selected to maintain the temperature of the heating element at the target temperature range effective for performing an endoscopic vessel harvesting procedure.

19. A method for making a direct current power control system for an endoscopic vessel harvesting system to provide controlled power to a therapeutic tool comprising a heating element, the method comprising:
connecting an input connection to a circuit board comprising a first power control circuit and a second power control circuit, the input connection configured to receive power from a power supply, the first power control circuit configured to supply constant output power to the heating element during a first time interval to heat the heating element to a target temperature, and the second power control circuit configured to supply pulsed output power to the heating element during a second time interval following the first time interval to maintain a temperature of the heating element within a target temperature range; and
connecting the circuit board to an output connection, the output connection configured to receive the constant output power and the pulsed output power to supply controlled power to the heating element of the therapeutic tool, wherein supplying of controlled power to the heating element comprises, in sequence, supplying the constant output power during the first time interval and then the pulsed output power during the second time interval to the heating element followed by a third time interval during which no power is supplied to the heating element to allow heat to dissipate from the heating element.

20. The method of claim 19, wherein the first power control circuit comprises a first one-shot pulse generator circuit.

21. The method of claim 19, wherein the second power control circuit comprises a second one-shot pulse generator circuit and an oscillator circuit, wherein an output of the second one-shot pulse generator circuit is connected to an input of the oscillator circuit.

22. The method of claim 19, further comprising connecting the first power control circuit and the second power control circuit to the circuit board before connecting the input connection to the circuit board.

23. The method of claim 19, further comprising:
encasing the circuit board and the output connection in a housing,
wherein connecting the input connection to the circuit board comprises connecting the circuit board to a distal end of a cable extending from the housing and connecting the input connection to a proximal end of the cable.

24. The method of claim 19, further comprising:
connecting a first logic gate to the circuit board so that the first logic gate is connected to the first power control circuit and the second power control circuit, wherein the first logic gate is configured to output only one of the constant output power from the first power control circuit or the pulsed output power from the second power control circuit at a time; and
connecting a second logic gate to the circuit board so that the second logic gate is connected to the first logic gate and a switch, wherein the second logic gate is configured to output the constant output power or the pulsed output power from the first logic gate only after the switch is closed.

25. An endoscopic vessel harvesting system, comprising:
a power supply;
a therapeutic tool comprising a heating element connected to a cutting element; and
a direct current power control system, comprising:
an input connection configured to receive power from the power supply;
a circuit board connected to the input connection, the circuit board configured to supply constant output power to the heating element during a first time interval to heat the heating element to a target temperature and to supply pulsed output power to the heating element during a second time interval following the first time interval to maintain a temperature of the heating element within a target temperature range; and
an output connection connected to the circuit board, the output connection configured to receive one of the constant output power or the pulsed output power at a time, and to supply controlled power to the heating element, wherein supplying of controlled power to the heating element comprises, in sequence, supplying the constant output power during the first time interval and then the pulsed output power during the second time interval to the heating element followed by a third time interval during which no power is supplied to the heating element in order to allow heat to dissipate from the heating element to avoid overheating the heating element.

26. The endoscopic vessel harvesting system of claim 25, wherein the circuit board comprises:
a first power control circuit connected to the input connection, the first power control circuit configured to supply the constant output power to the heating element during the first time interval to heat the heating element to the target temperature; and
a second power control circuit connected to the input connection, the second power control circuit configured to supply the pulsed output power to the heating element during the second time interval to maintain a temperature of the heating element within a target temperature range.
